(19) United States
(12) Patent Application Publication
(10) Pub. No.: US 2005/0014732 A1
(43) Pub. Date: Jan. 20, 2005

(54) Combination of an aldosterone receptor antagonist and an anti-diabetic agent

(75) Inventors: Eric Arthur Gulve, St. Louis, MO (US); Ellen Garwitz McMahon, St. Louis, MO (US)

Correspondence Address: HARNESS, DICKEY & PIERCE, P.L.C., P.O. BOX 8910, RESTON, VA, 20195, US

(73) Assignee: Pharmacia Corporation, St. Louis, MO (US)

(21) Appl. No.: 10/767,839

(22) Filed: Jan. 30, 2004

Related U.S. Application Data
(60) Provisional application No. 60/454,326, filed on Mar. 14, 2003.

(51) Int. Cl. A61K 31/56

(52) U.S. Cl. 514/171

(57) ABSTRACT

A combination therapy comprising a therapeutically-effective amount of an aldosterone receptor antagonist and a therapeutically-effective amount of an anti-diabetic agent is described for treatment of circulatory disorders, including cardiovascular disorders such as hypertension, congestive heart failure, cirrhosis and ascites. Preferred antidiabetic agents are those compounds having high potency and oral or parenteral bioavailability. Preferred aldosterone receptor antagonists are 20-spiroxane steroidal compounds characterized by the presence of a 9α,11α-substituted epoxy moiety.

### CROSS-REFERENCE TO RELATED APPLICATION

This non-provisional application claims priority to provisional Application No. 60/454,326, filed March 14, 2003, incorporated herein by reference in its entirety.

### FIELD OF THE INVENTION

Combinations of an aldosterone receptor antagonist and anti-diabetic agents are described for use in treatment of circulatory disorders, including cardiovascular diseases such as hypertension, cardiovascular disease, renal dysfunction, cerebrovascular disease, vascular disease, retinopathy, neuropathy, hyperglycemia, hyperinsulinemia and insulin resistance, edema, endothelial dysfunction, and baroreceptor dysfunction. Of particular interest are therapies using a steroidal aldosterone receptor antagonist compound in combination with an anti-diabetic agent.

### BACKGROUND OF THE INVENTION

Aldosterone

Aldosterone is the body's most potent known mineralocorticoid hormone. As connoted by the term mineralocorticoid, this steroid hormone has mineral-regulating activity. It promotes sodium (Na⁺) reabsorption not only in the kidney, but also from the lower gastrointestinal tract and salivary and sweat glands, each of which represents classic aldosterone-responsive tissues. Aldosterone increases sodium and water reabsorption in the distal nephron and promotes potassium (K⁺) and magnesium (Mg²⁺) excretion.

Aldosterone also can produce responses in nonepithelial cells. In fact, aldosterone receptors have been recently identified in brain tissue, heart tissue and blood vessels. These aldosterone-mediated responses can have adverse consequences on the structure and function of the cardiovascular system and other tissues and organs. Hence, aldosterone can contribute to organ damage for multiple reasons.

Aldosterone Receptor Antagonists

The effects of aldosterone can be blocked through the use of an aldosterone receptor antagonist. The only aldosterone receptor antagonist that is commercially available at this time is spironolactone (also known as ALDACTONE®). Spironolactone is indicated for the management of essential hypertension, primary aldosteronism, hypokalemia, and edematous conditions such as congestive heart failure, cirrhosis of the liver and nephrotic syndrome. The United States Pharmacopeia, 21^{st} Revision (16^{th} Edition), United States Pharmacopeial Convention, Inc., Rockville, Md. (1985) and each and every subsequent edition to date thereof. The administration of spironolactone to severe heart failure patients was evaluated in the Randomized Aldactone Evaluation Study (RALES). RALES was a randomized, double-blinded, placebo-controlled trial that enrolled participants who had severe heart failure and a left ventricular ejection fraction of no more than 35% and who were receiving standard therapy, including an angiotensin-converting enzyme inhibitor, a loop diuretic, and, in some cases, digoxin and a beta-blocker. The RALES subjects treated with spironolactone had a statistically significant reduction in mortality and incidence of hospitalization relative to placebo-treated subjects. New England Journal of Medicine 341, 709-717 (1999). A class of steroidal-type aldosterone receptor antagonists exemplified by epoxy-containing spirolactone derivatives is described in U.S. Pat. No. 4,559,332 issued to Grob et al. This patent describes 9α,11α-epoxy-containing spirolactone derivatives as aldosterone receptor antagonists that are useful for the treatment of hypertension, cardiac insufficiency and cirrhosis of the liver. One of the epoxy-steroidal aldosterone receptor antagonist compounds described in U.S. Pat. 4,559,332 is eplerenone (also known as epoxymexrenone). Eplerenone is an aldosterone receptor antagonist that has a greater selectivity for the aldosterone receptor than does, for example, spironolactone.

WO01/95892 and WO01/95893 describe methods for the treatment of aldosterone-mediated pathogenic effects in a subject using an aldosterone receptor antagonist (including spironolactone and/or eplerenone).

WO02/09683 describes methods of using an aldosterone receptor antagonist (including eplerenone and/or spironolactone) for the treatment of inflammation in a subject.

Antidiabetic Agents

A plethora of agents are known for treatment of diabetes or syndromes or conditions related to diabetes. For example, Dr. Salim Yusef et al's article in The New England Journal of Medicine, Vol. 342, No. 3, Jan. 20, 2000, pp 145-153, describes the effects of an angiotensin-converting-enzyme inhibitor, ramipril, in patients (including diabetics) who were at high risk for cardiovascular events.

An article by Robert C. Turner, et al. appearing in The Lancet Vol. 352, Sep. 12, 1998, pp 837-853, compares the effects of intensive blood-glucose control with either sulphonylureas or insulin with conventional treatment in patients with type 2 diabetes.

An article by Dr. James I. Cleeman appearing in JAMA, Vol. 285, No. 19, May 16, 2001, pp. 2486-2497, describes the detection and treatment of high blood cholesterol in adults with diabetes, a group at particularly high risk for cardiovascular morbidity and mortality at any given blood cholesterol level.

The treatment of cardiovascular and renal risk factors in a patient with diabetes, hypertension, left ventricular hypertrophy, and diabetic nephropathy is described in an article by James R. Sowers and Steven Haffner appearing in Hypertension, Vol. 40, 2002, pp 781-788. A rationale for the therapy is discussed on page 784 entitled “Renin-Angiotensin System an Antihypertensive Therapy” based on prior clinical studies.

An article by Bo Isomaa describes the relationship between the Metabolic Syndrome and excess cardiovascular mortality/morbidity. “Cardiovascular Morbidity and Mortality Associated with Metabolic Syndrome” Diabetes Care, Vo. 24, No. 4, April 2001.

Combination Therapy

Therapies comprising the administration of an aldosterone receptor antagonist in combination with several other pharmacologically active compounds have been reported in the literature.

WO 96/40255, incorporated herein in its entirety, discloses a combination treatment therapy utilizing an epoxy-steroidal aldosterone receptor antagonist and an angiotensin II antagonist for treating cardiac fibrosis.

WO 96/40257, incorporated herein in its entirety, discloses a combination treatment therapy utilizing an epoxy-steroidal aldosterone receptor antagonist and an angiotensin II antagonist for treating congestive heart failure.

Perez et al., WO 00/27380, incorporated herein in its entirety, discloses a combination treatment therapy utilizing an angiotensin converting enzyme inhibitor and an aldosterone receptor antagonist for reducing morbidity and mortality resulting from cardiovascular disease.

Alexander et al., WO 00/51642, incorporated herein in its entirety, discloses a combination treatment therapy utilizing an angiotensin converting enzyme inhibitor and an epoxy-steroidal aldosterone receptor antagonist for treating cardiovascular disease.

Alexander et al., WO 02/09760, incorporated herein in its entirety, discloses a combination therapy utilizing an epoxy-steroidal aldosterone receptor antagonist and a beta-adrenergic antagonist for treating circulatory disorders, including cardiovascular disorders such as hypertension, congestive heart failure, cirrhosis and ascites.

Schuh, WO 02/09761, incorporated herein in its entirety, discloses a combination treatment therapy utilizing an epoxy-steroidal aldosterone receptor antagonist and a calcium channel blocker for treating hypertension, congestive heart failure, cirrhosis and ascites.

Rocha, WO 02/09759, incorporated herein in its entirety, discloses a combination treatment therapy utilizing an epoxy-steroidal aldosterone receptor antagonist and a cyclooxygenase-2 inhibitor for treating inflammation-related cardiovascular disorders.

J. B. Marks, et al. “Cardiovascular Risk in Diabetes A Brief Review,” Journal of Diabetes and Its Complications 14 (2000) 108-115 focuses on known modifiable risk factors for cardiovascular disease associated with diabetes, potential targets for primary and secondary prevention.

Improved drug therapies for the treatment of subjects suffering from or susceptible to a pathological condition are highly desirable. In particular, there still is a need for drug therapies that (1) provide better control over pathological conditions, (2) further reduce pathological risk factors, (3) provide improved treatment and/or prevention of pathological conditions, (4) are effective in a greater proportion of subjects suffering from or susceptible to a pathological condition, particularly in those subjects who do not satisfactorily respond to conventional drug therapies, and/or (5) provide an improved side-effect profile relative to conventional drug therapies.

For example, improved drug therapies for the treatment of subjects suffering from or susceptible to a cardiovascular-related condition are highly desirable. In particular, there still is a need for drug therapies that (1) provide better control over cardiovascular-related conditions, (2) further reduce cardiovascular-related risk factors, (3) provide improved treatment and prevention of cardiovascular-related conditions, (4) are effective in a greater proportion of subjects suffering from or susceptible to a cardiovascular-related condition, particularly in those subjects who do not satisfactorily respond to conventional drug therapies, and/or (5) provide an improved side-effect profile relative to conventional drug therapies.

### BRIEF SUMMARY OF THE INVENTION

A combination therapy comprising a therapeutically-effective amount of an aldosterone receptor antagonist and a therapeutically-effective amount of an anti-diabetic agent is useful to treat circulatory disorders, including cardiovascular disorders such as hypertension, cardiovascular disease, renal dysfunction, liver disease, cerebrovascular disease, vascular disease, retinopathy, neuropathy, hyperglycemia, hyperinsulinemia and insulin resistance, edema, endothelial dysfunction, and baroreceptor dysfunction.

A method for the prophylaxis or treatment of a cardiovascular-related condition, the method comprising administering to a subject susceptible to or afflicted with such condition a first amount of an aldosterone receptor antagonist and a second amount of an anti-diabetic agent, wherein the first amount of the aldosterone receptor antagonist and the second amount of the anti-diabetic agent together comprise a therapeutically-effective amount of the aldosterone receptor antagonist and anti-diabetic agent.

Unless indicated otherwise, the following definitions or terms are used throughout this specification:

The terms “treat,” “treatment” or “treating” include the administration, to a person in need of or susceptible to a cardiovascular-related condition, of an amount of an aldosterone antagonist and anti-diabetic agent in a combination that will prevent the onset of, inhibit or reverse development of a pathological cardiovascular condition.

The terms “prevent,” “prevention” or “preventing” includes either preventing the onset of one or more clinically evident cardiovascular-related conditions altogether or preventing the onset of a preclinically evident stage of one or more cardiovascular-related conditions in individuals. This includes prophylactic treatment of those at risk of developing one or more cardiovascular-related conditions.

The phrase “therapeutically-effective” is intended to qualify the amount of the two agents given in combination which will achieve the goal of improvement in cardiovascular-related condition severity and the frequency of incidence, while avoiding adverse side effects.

The term “subject” for purposes of treatment includes any human or animal subject who is susceptible to or suffering from one or more cardiovascular-related conditions, and preferably is a human subject. The subject, for example, may be at risk due to diet, exposure to bacterial or viral infection, having common markers present, being genetically predisposed to one or more cardiovascular-related conditions, and the like.

The term “insulin” as used herein includes, but is not limited to, any currently known wild-type or mutant forms of injectable insulin, oral insulin, inhalational insulin or other types of formulations of insulin. See Remington's Pharmaceutical Sciences, 16^{th} Ed., Arthur Osol (Editor), Mack Publishing Co., Easton, Pa. (1980) and each and every subsequent edition to date thereof. See also The Merck Index, 12^{th} Edition, S. Budavari (Editor), Merck & Co., Inc., Whitehouse Station, N.J. (1996) and each and every subsequent edition to date thereof.

A drug (as disclosed herein such as an anti-diabetic agent) includes its regular and slow-release formulations (e.g., metformin versus metformin HCl extended-release tablets—once daily doses).

### DETAILED DESCRIPTION OF THE INVENTION

Aldosterone Receptor Antagonists

The term “aldosterone receptor antagonist” denotes a compound capable of binding to an aldosterone receptor, as a competitive inhibitor of the action of aldosterone itself at the receptor site, so as to modulate the receptor-mediated activity of aldosterone.

The aldosterone receptor antagonists used in the combinations and methods of the present invention generally are spirolactone-type steroidal compounds. The term “spirolactone-type” is intended to characterize a structure comprising a lactone moiety attached to a steroid nucleus, typically at the steroid “D” ring, through a spiro bond configuration. A subclass of spirolactone-type aldosterone receptor antagonist compounds consists of epoxy-steroidal aldosterone receptor antagonist compounds such as eplerenone. Another subclass of spirolactone-type antagonist compounds consists of non-epoxy-steroidal aldosterone receptor antagonist compounds such as spironolactone.

The epoxy-steroidal aldosterone receptor antagonist compounds used in the combinations and method of the present invention generally have a steroidal nucleus substituted with an epoxy-type moiety. The term “epoxy-type” moiety is intended to embrace any moiety characterized in having an oxygen atom as a bridge between two carbon atoms, examples of which include the following moieties:

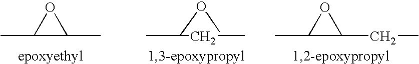

The term “steroidal”, as used in the phrase “epoxy-steroidal”, denotes a nucleus provided by a cyclopenteno-phenanthrene moiety, having the conventional “A”, “B”, “C” and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system. The phrase “epoxy-steroidal” is intended to embrace a steroidal nucleus having one or a plurality of epoxy-type moieties attached thereto.

Epoxy-steroidal aldosterone receptor antagonists suitable for use in the present combinations and methods include a family of compounds having an epoxy moiety fused to the “C” ring of the steroidal nucleus. Especially preferred are 20-spiroxane compounds characterized by the presence of a 9α,11α-substituted epoxy moiety. Compounds 1 through 11, below, are illustrative 9α,11α-epoxy-steroidal compounds that may be used in the present methods. A particular benefit of using epoxy-steroidal aldosterone receptor antagonists, as exemplified by eplerenone, is the high selectivity of this group of aldosterone receptor antagonists for the mineralocorticoid receptor. The superior selectivity of eplerenone results in a reduction in side effects, that can be caused by aldosterone receptor antagonists that exhibit non-selective binding to other steroid receptors, such as androgen and progesterone receptors.

These epoxy steroids may be prepared by procedures described in Grob et al., U.S. Pat. No. 4,559,332. Additional processes for the preparation of 9,11-epoxy steroidal compounds and their salts are disclosed in Ng et al., WO97/21720 and Ng et al., WO98/25948.

TABLE I
Aldosterone Receptor Antagonist

| Compound # | Structure | Name |
|---|---|---|
| 1 | 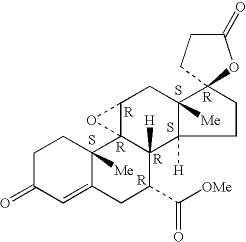 | Pregn-4-ene-7,21-dicarboxylic acid, 9,11-epoxy- 17-hydroxy-3-oxo-,γ-lactone, methyl ester, (7α, 11α, 17β)- |
| 2 | 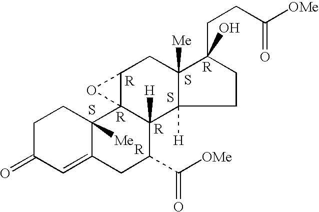 | Pregn-4-ene-7,21-dicarboxylic acid, 9,11-epoxy- 17-hydroxy-3-oxo-,dimethyl ester, (7α, 11α, 17β)- |
| 3 | 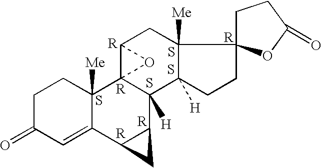 | 3′H-cyclopropa[6, 7]pregna-4,6-diene-21-carboxylic acid, 9,11-epoxy-6,7-dihydro-17-hydroxy-3-oxo-, γ-lactone, (6β, 7β, 11α, 17β)- |
| 4 | 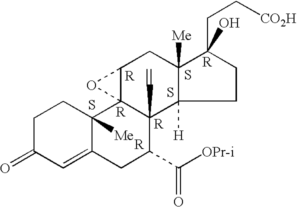 .K⁺ | Pregn-4-ene-7,21-dicarboxylic acid, 9,11-epoxy-17- hydroxy-3-oxo-,7-(1-methylethyl) ester, monopotassium salt, (7α, 11α, 17β)- |
| 5 | 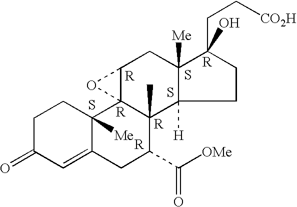 .K⁺ | Pregn-4-ene-7,21-dicarboxylic acid, 9,11-epoxy-17- hydroxy-3-oxo-,7-methylethyl) ester, monopotassium salt, (7α, 11α, 17β)- |
| 6 | 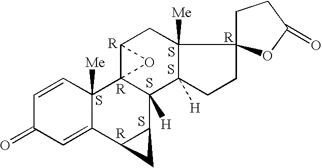 | 3′H-cyclopropa[6, 7]pregna-1,4,6-triene-21-carboxylic acid, 9,11-epoxy-6,7-dihydro-17-hydroxy-3-oxo-, γ-lactone(6γ, 7γ, 11α)- |
| 7 | 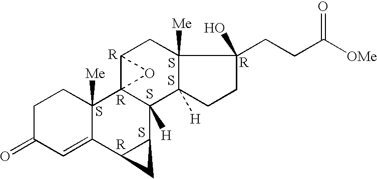 | 3′H-cyclopropa[6, 7]pregna-4,6-diene-21-carboxylic acid, 9,11-epoxy-6,7-dihydro-17-hydroxy-3-oxo-, methyl ester,(6β, 7β, 11α, 17β)- |
| 8 | 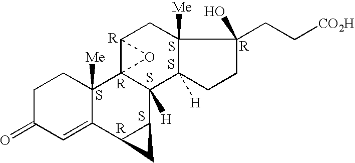 | 3′H-cyclopropa[6, 7]pregna-4,6-diene-21-carboxylic acid, 9,11-epoxy-6,7-dihydro-17-hydroxy-3-oxo-, monopotassium salt, (6β, 7β, 11α, 17β)- |
| 9 | 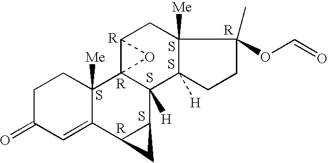 | 3′H-cyclopropa[6, 7]pregna-1,4,6-triene-21-carboxylic acid, 9,11-epoxy-6,7-dihydro-17-hydroxy-3-oxo-,β- lactone (6β, 7β, 11α, 17β)- |
| 10 | 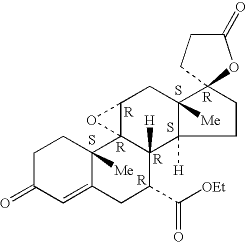 | Pregn-4-ene-7,21-dicarboxylic acid, 9,11-epoxy- 17-hydroxy-3-oxo-,β-lactone, ethyl ester, (7α, 11α, 17β)- |
| 11 | 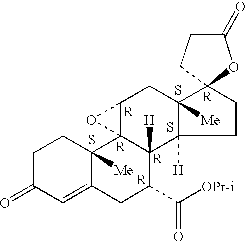 | Pregn-4-ene-7,21-dicarboxylic acid, 9,11-epoxy- 17-hydroxy-3 -oxo-,β-lactone,1-methylethyl ester (7α, 11α, 17β)- |

Of particular interest is the compound eplerenone (also known as epoxymexrenone) which is compound 1 as shown above. Eplerenone is an aldosterone receptor antagonist with a greater selectivity for aldosterone receptors than, for example, spironolactone. Selection of eplerenone as the aldosterone receptor antagonist in the present method would be beneficial to reduce certain side-effects such as gynecomastia, menstrual irregularities and impotence that occur with use of aldosterone receptor antagonists having less selectivity.

Non-epoxy-steroidal aldosterone receptor antagonists suitable for use in the present methods include a family of spirolactone-type compounds defined by Formula I: 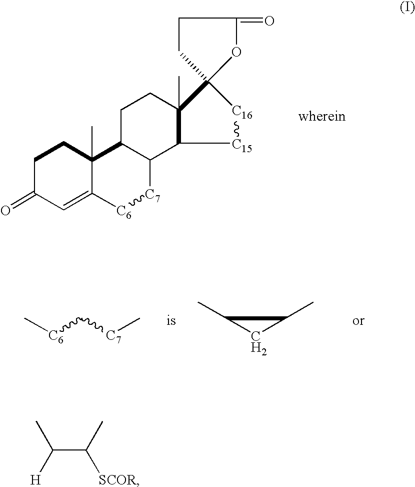
wherein R is lower alkyl of up to 5 carbon atoms, and

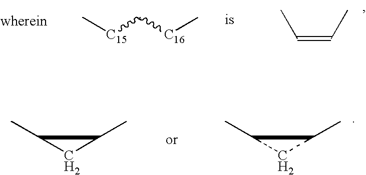

Lower alkyl residues include branched and unbranched groups, preferably methyl, ethyl and n-propyl.

Specific compounds of interest within Formula I are the following:
7α-acetylthio-3-oxo-4,15-androstadiene-[17(β-1′)-spiro-5′]perhydrofuran-2′-one;
3-oxo-7α-propionylthio-4,15-androstadiene-[17((β-1′)-spiro-5′]perhydrofuran-2′-one;
6β,7β-methylene-3-oxo4,15-androstadiene-[17((β-1′)-spiro-5′]perhydrofuran-2′-one;
15α,16α-methylene-3-oxo-4,7α-propionylthio4-androstene[17(β-1′)-spiro-5′]perhydrofuran-2′-one;
6β,7β,15α,16α-dimethylene-3-oxo-4-androstene[17(β-1′)-spiro-5′]-perhydrofuran-2′-one;
7α-acetylthio-15β,16β-Methylene-3-oxo-4-androstene-[17(β-1′)-spiro-5′]perhydrofuran-2′-one;
15β,16β-methylene-3-oxo-7β-propionylthio-4-androstene-[17(β-1′)-spiro-5′]perhydrofuran-2′-one; and
6β,7β,15β,16β-dimethylene-3-oxo-4-androstene-[17(β-1′)-spiro-5′]perhydrofuran-2′-one.

Methods to make compounds of Formula I are described in U.S. Pat. No. 4,129,564 to Wiechart et al. issued on 12 Dec. 1978.

Another family of non-epoxy-steroidal compounds of interest is defined by Formula II: 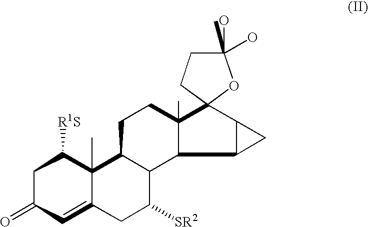
wherein R¹ is C₁₋₃-alkyl or C₁₋₃ acyl and R² is H or C₁₋₃-alkyl.

Specific compounds of interest within Formula II are the following:
1α-acetylthio-15β,16β-methylene-7α-methylthio-3-oxo-17α-pregn-4-ene-21,17-carbolactone; and
15β,16β-methylene-1α,7α-dimethylthio-3-oxo-17α-pregn-4-ene-21,17-carbolactone.

Methods to make the compounds of Formula II are described in U.S. Pat. No. 4,789,668 to Nickisch et al. which issued 6 Dec. 1988.

Yet another family of non-epoxy-steroidal compounds of interest is defined by a structure of Formula III: 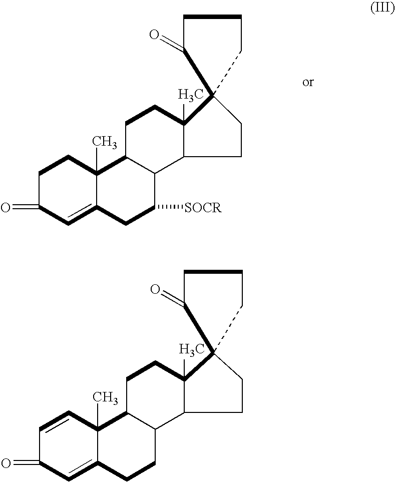
wherein R is lower alkyl, with preferred lower alkyl groups being methyl, ethyl, propyl and butyl. Specific compounds of interest include:
3β,21-dihydroxy-17α-pregna-5,15-diene-17-carboxylic acid(-lactone;
3β,21-dihydroxy-17α-pregna-5,15-diene-17-carboxylic acid(-lactone 3-acetate;
3β,21-dihydroxy-17α-pregn-5-ene-17-carboxylic acid(-lactone;
3β,21-dihydroxy-17α-pregn-5-ene-17-carboxylic acid(-lactone 3-acetate;
21-hydroxy-3-oxo-17α-pregn-4-ene-17-carboxylic acid(-lactone;
21-hydroxy-3-oxo-17α-pregna-4,6-diene-17-carboxylic acid(-lactone;
21-hydroxy-3-oxo-17α-pregna-1,4-diene-17-carboxylic acid(-lactone;
7α-acylthio-21-hydroxy-3-oxo-17α-pregn-4-ene-17-carboxylic acid(lactone; and
7α-acetylthio-21-hydroxy-3-oxo-17α-pregn-4-ene-17-carboxylic acid(-lactone.

Methods to make the compounds of Formula III are described in U.S. Pat. No. 3,257,390 to Patchett which issued 21 Jun. 1966.

Still another family of non-epoxy-steroidal compounds of interest is represented by Formula IV: 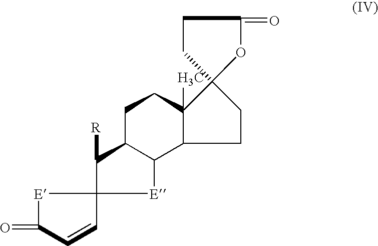
wherein E′ is selected from the group consisting of ethylene, vinylene and (lower alkanoyl)thioethylene radicals, E″ is selected from the group consisting of ethylene, vinylene, (lower alkanoyl)thioethylene and (lower alkanoyl)thiopropylene radicals; R is a methyl radical except when E′ and E″ are ethylene and (lower alkanoyl) thioethylene radicals, respectively, in which case R is selected from the group consisting of hydrogen and methyl radicals; and the selection of E′ and E″ is such that at least one (lower alkanoyl)thio radical is present.

A preferred family of non-epoxy-steroidal compounds within Formula IV is represented by Formula V:

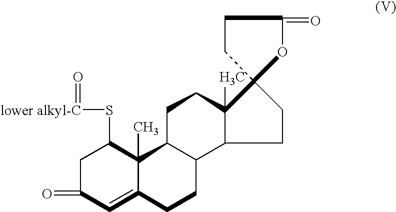

A more preferred compound of Formula V is
1-acetylthio-17α-(2-carboxyethyl)-17β-hydroxy-androst-4-en-3-one lactone.

Another preferred family of non-epoxy-steroidal compounds within Formula IV is represented by Formula VI:

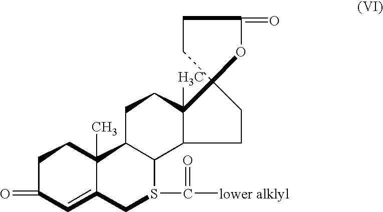

More preferred compounds within Formula VI include the following:
7α-acetylthio-17α-(2-carboxyethyl)-17β-hydroxy-androst-4-en-3-one lactone;
7β-acetylthio-17α-(2-carboxyethyl)-17β-hydroxy-androst-4-en-3-one lactone;
1α,7α-diacetylthio-17α-(2-carboxyethyl)-17β-hydroxy-androsta-4,6-dien-3-one lactone;
7α-acetylthio-17α-(2-carboxyethyl)-17β-hydroxy-androsta-1,4-dien-3-one lactone;
7α-acetylthio-17α-(2-carboxyethyl)-17β-hydroxy-19-norandrost-4-en-3-one lactone; and
7α-acetylthio-17α-(2-carboxyethyl)-17β-hydroxy-6α-methylandrost-4-en-3-one lactone;

In Formulae IV-VI, the term “alkyl” is intended to embrace linear and branched alkyl radicals containing one to about eight carbons. The term “(lower alkanoyl)thio” embraces radicals of the formula lower alkyl

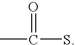

Of particular interest is the compound spironolactone having the following structure and formal name: 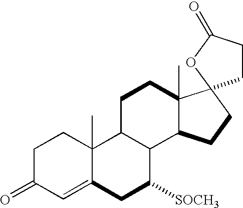
“spironolactone”: 17-hydroxy-7α-mercapto-3-oxo-17α-pregn-4-ene-21-carboxylic acid γ-lactone acetate.

Methods to make compounds of Formulae IV-VI are described in U.S. Pat. No. 3,013,012 to Cella et al. which issued 12 Dec. 1961. Spironolactone is sold by G.D. Searle & Co., Skokie, Ill., under the trademark “ALDACTONE”, in tablet dosage form at doses of 25 mg, 50 mg and 100 mg per tablet.

Another family of steroidal aldosterone receptor antagonists is exemplified by drospirenone, [6R-(6alpha,7alpha,8beta,9alpha,10beta,13beta,14alpha,15alpha,16alpha,17beta)]-1,3′,4′,6,7,8,9,10,11,12,13,14,15,16,20,21-hexadecahydro-10,13-dimethylspiro[17H-dicyclopropa[6,7:15,16]cyclopenta[a]phenanthrene-17,2′(5′H)-furan]-3,5′(2H)-dione, CAS registration number 67392-87-4. Methods to make and use drospirenone are described in patent GB 1550568 1979, priority DE 2652761 1976.

Anti-Diabetic Agents

Anti-diabetic agents include oral anti-diabetic agents; hypoglycemia treatment agents, and insulins. Tables 2-10, below, describe various agents, which may be used in the combination therapy. Each published patent document listed in the tables describes the chemical preparation of the associated anti-diabetic agent as well as the biological properties of such compound. The content of each of these patent documents is incorporated herein by reference.

One embodiment includes anti-diabetic agents and drugs of Table 2.

TABLE 2

| | Chemical | |
|---|---|---|
| | Abstract | Reference to |
| Name of Agent | Number | Source of Compound |
| Acarbose | 56180-94-0 | Carbohydrate Research |
| | | (1989), Vol. 189, |
| | | pages 309-22 |
| Acetohexamide | 968-81-0 | FR 1588266 |
| | | Issued: Apr. 10, 1970 |
| Buformin | 692-13-7 | Nippon Kagaku Kaishi |
| | | (1993), (8), pages |
| | | 952-956 |
| 1-Butyl-3- | 4618-41-1 | WO 2000/061541 |
| metanilylurea | | Issued: Oct. 19, 2000 |
| Carbutamide | 339-43-5 | J. Chem. Soc. C (1967), |
| | | (8), pages 701-702 |
| Chlorpropamide | 94-20-2 | JP 43007938 |
| | | Issued: Mar. 26, 1968 |
| Ciglitazone | 74772-77-3 | Chem. Pharm. Bull. |
| | | (1982), Vol. 30(10), |
| | | pages 3580-3600 |
| Glibornuride | 26944-48-9 | U.S. Pat. No. 3832397 |
| | | Issued: Aug. 27, 1974 |
| Gliclazide | 21187-98-4 | JP 06041073 |
| | | Issued: Feb. 15, 1994 |
| Glimepiride | 93479-97-1 | WO 01/05354 |
| | | Issued: Jan. 25, 2001 |
| Glipizide | 29094-61-9 | DE 2012138 |
| | | Issued: Oct. 01, 1970 |
| Gliquidone | 33342-05-1 | DE 2011126 |
| | | Issued: Oct. 07, 1971 |
| Glisoxepid | 25046-79-1 | U.S. Pat. No. 3668215 |
| | | Issued: Jun. 06, 1972 |
| Glyburide | 10238-21-8 | DE 1283837 |
| | | Issued: Nov. 28, 1968 |
| Glybuthiazole | 535-65-9 | Ann. Pharm. France |
| | | (1966), Vol. 24(9-10), |
| | | pages 593-605 |
| Glybuzole | 1492-02-0 | DE 4336159 |
| | | Issued: Apr. 27, 1995 |
| Glyhexamide | 451-71-8 | Chim. Ther. (1973), |
| | | Vol. 8(6), pages |
| | | 659-668 |
| Glymidine | 339-44-6 | U.S. Pat. No. 3288793 |
| | | Issued: Nov. 29, 1966 |
| Glypinamide | 1228-19-9 | FR 1458907 |
| | | Issued: Nov. 18, 1966 |
| Metformin | 657-24-9 | DE 2444532 |
| | | Issued: Mar. 27, 1975 |
| Miglitol | 72432-03-2 | JP 54106477 |
| | | Issued: Aug. 21, 1979 |
| Nateglinide | 105816-04-4 | J. Med. Chem. (1989), |
| | | Vol. 32(7), pages |
| | | 1436-1441 |
| Phenbutamide | 3149-00-6 | FR 1552925 |
| | | Issued: Jan. 10, 1969 |
| Phenformin | 114-86-3 | Methods Enzymol. (1982), |
| | | Vol. 84(Immunochem. Tech., |
| | | Part D), pages 577-585 |
| Pioglitazone | 111025-46-8 | EP 193256 |
| | | Issued: Sep. 03, 1986 |
| Proinsulin | 9035-68-1 | WO 01/072959 |
| | | Issued: Oct. 04, 2001 |
| Repaglinide | 135062-02-1 | WO 93/00337 |
| | | Issued: Jan. 07, 1993 |
| Rosiglitazone | 122320-73-4 | EP 306228 |
| | | Issued: Mar. 08, 1989 |
| Tolazamide | 1156-19-0 | NL 6603398 |
| | | Issued: Sep. 19, 1966 |
| Tolbutamde | 64-77-7 | J. Chem. Soc. C (1967) |
| | | (8), pages 701-702 |
| Tolcyclamide | 664-95-9 | NL 6603398 |
| | | Issued: Sep. 19, 1966 |
| Troglitazone | 97322-87-7 | WO 97/43283 |
| | | Published: Nov. 20, 1997 |

Another embodiment includes anti-diabetic agents and drugs of Table 3.

TABLE 3

| | Chemical | |
|---|---|---|
| | Abstract | Reference to |
| Name of Agent | Number | Source of Compound |
| Acipimox | 51037-30-0 | DE 2319834 |
| | | Issued: Nov. 15, 1973 |
| Amiloride | 2609-46-3 | FR 1525692 |
| | | Issued: May 17, 1968 |
| Benfluorex | 23602-78-0 | ES 474498 |
| | | Issued: Apr. 16, 1979 |
| BTS 67582 | 161748-40-9 | Idrugs (1999), Vol. |
| | | 2(4), pages 255-359 |
| Clofibrate | 637-07-0 | J. Med. Chem. (1974), |
| | | Vol. 17(1), pages |
| | | 108-112 |
| Darglitazone | 141200-24-0 | J. Med. Chem. (1992), |
| | | Vol. 35(10), pages |
| | | 1853-1864 |
| Dehydroepi- | 53-43-0 | Tetrahedron Lett. (1997), |
| androsterone | | Vol. 38(13), pages |
| | | 2253-2256 |
| Efaroxan | 89197-32-0 | WO 00/15624 |
| | | Issued: Mar. 23, 2000 |
| Emiglitate | 80879-63-6 | International J. Clin. |
| | | Pharm., Therapy, and |
| | | Tox., (1987), Vol. 25(9), |
| | | pages 483-488 |
| Englitazone | 109229-58-5 | WO 86/07056 |
| | | Issued: Dec. 04, 1986 |
| Epalrestat | 82159-09-9 | Huandong Shifan Daxue |
| | | Xuebao, Ziran Kexueban |
| | | (1999), (3), pages |
| | | 104-106 |
| Exendin-4 | 141732-76-5 | J. Biol. Chem. (1993), |
| | | Vol. 268(26), pages |
| | | 19650-19655 |
| Fenfluramine | 458-24-2 | Bull. Soc. Chim. Fr. |
| | | (1993), Vol. 130(4), |
| | | pages 459-466 |
| Fidarestat | 136087-85-9 | JP 2001302670 |
| | | Issued: Oct. 31, 2001 |
| Glisentide | 32797-92-5 | DE 2146861 |
| | | Issued: Mar. 30, 1972 |
| Glisolamide | 24477-37-0 | DE 1670807 |
| | | Issued: Aug. 07, 1975 |
| Glucagon-like | 89750-14-1 | WO 00/34331 |
| peptide I | | Issued: Jun. 15, 2000 |
| Glyclopyramide | 631-27-6 | Chem. Pharm. Bull. |
| | | (1969), Vol. 17(8), |
| | | pages 1535-1540 |
| Insulinotropin | 118549-37-4 | WO 01/98331 |
| | | Issued: Dec. 27, 2001 |
| Leptin | 169494-85-3 | CN 1273248 |
| | | Issued: Dec. 15, 2000 |
| Meglitinide | 54870-28-9 | DE 2500157 |
| | | Issued: Jul. 22, 1976 |
| Minalrestat | 129688-50-2 | EP 365324 |
| | | Issued: Apr. 25, 1990 |
| Mitiglinide | 145375-43-5 | WO 99/01430 |
| | | Issued: Jan. 14, 1999 |
| Orlistat | 96829-58-2 | Chem. Commun. (Cambridge) |
| | | (1999), (17), |
| | | pages 1743-1744 |
| Pramlintide | 151126-32-8 | WO 93/10146 |
| | | Issued: May 27, 1993 |
| Reglitazar | 170861-63-9 | WO 95/18125 |
| | | Issued: Jul. 06, 1995 |
| Sibutramine | 106650-56-0 | Zhongguo Yaowu |
| | | Huaxue Zazhi (2000), |
| | | Vol. 10(2), pages |
| | | 129-130, 140 |
| Sorbinil | 68367-52-2 | J. Org. Chem. (1987), |
| | | Vol. 52(16), pages |
| | | 3587-3591 |
| Theophyllin | 58-55-9 | Chem. Eng. World (1998), |
| | | Vol. 33(11), pages |
| | | 110-112 |
| Voglibose | 83480-29-9 | EP 56194 |
| | | Issued: Jul. 21, 1982 |
| Zenarestat | 112733-06-9 | Chem. Express (1993). |
| | | Vol. 8(9), pages |
| | | 761-764 |
| Zopolrestat | 110703-94-1 | J. Med. Chem. (1991), |
| | | Vol. 34(1), pages |
| | | 108-122 |

Another embodiment includes developmental anti-diabetic agents and drugs of Table 4.

TABLE 4

| | Chemical | Reference to |
|---|---|---|
| Name of Agent | Abstract Number | Source of Compound |
| AC 2993 | 335149-21-8 | WO 2001/027107 |
| | | Issued: Apr. 19, 2001 |
| AJ 9677 | 244081-42-3 | JP 11255743 |
| | | Issued: Sep. 21, 1999 |
| AS 3201 | 147254-64-6 | EP 520320 |
| | | Issued: Dec. 30, 1992 |
| Arzoxifene | 182133-25-1 | U.S. Pat. No. 5723474 |
| | | Issued: Mar. 03, 1998 |
| BAY W1807 | 252721-95-2 | Protein Sci. (1999), |
| | | Vol. 8(10), pages |
| | | 1930-1945 |
| BL 11282 | 227798-41-6 | EP 924209 |
| | | Issued: Jun. 23, 1999 |
| BM 170744 | 221564-97-2 | Cardiovasc. Drug Rev. |
| | | (1999), Vol. 17(3), |
| | | pages 246-264 |
| BRL 35135 | 86615-96-5 | U.S. Pat. No. 5442118 |
| | | Issued: Aug. 15, 1995 |
| BRL 37344 | 90730-96-4 | U.S. Pat. No. 5442118 |
| | | Issued: Aug. 15, 1995 |
| BTA 188 | 330600-86-7 | WO 01/37837 |
| | | Issued: May 31, 2001 |
| BTS 67582 | 161748-40-9 | Idrugs (1999), |
| | | Vol. 2(4), pages |
| | | 355-359 |
| CD 3127 | 153559-76-3 | J. Med. Chem. (1995), |
| | | Vol. 38(16), pages |
| | | 3146-3155 |
| CL 316243 | 138908-40-4 | U.S. Pat. No. 5061727 |
| | | Issued: Oct. 29, 1991 |
| DRF 2189 | 172647-53-9 | EP 676398 |
| | | Issued: Oct. 11, 1995 |
| DRF 2725 | 222834-30-2 | WO 00/50414 |
| | | Issued: Aug. 31, 2000 |
| Farglitazar | 196808-45-4 | J. Med. Chem. (1998), |
| | | Vol. 41(25), pages |
| | | 5020-5036 |
| GW 1929 | 196808-24-9 | J. Med. Chem. (1998), |
| | | Vol. 41(25), pages |
| | | 5020-5036 |
| GW 2331 | 190844-95-2 | WO 00/08002 |
| | | Issued: Feb. 17, 2000 |
| GW 7845 | 196809-22-0 | WO 97/31907 |
| | | Issued: Sep. 04, 1997 |
| KAD 1229 | 145525-41-3 | Chem. Pharm. Bull. |
| | | (1998), Vol. 46(2), |
| | | pages 337-340 |
| L 783281 | 78860-34-1 | EP 1136071 |
| | | Issued: Sep. 26, 2001 |
| L 805645 | 209808-51-5 | WO 98/27974 |
| | | Issued: Jul. 02, 1998 |
| LG 100754 | 180713-37-5 | WO 97/12853 |
| | | Issued: Apr. 10, 1997 |
| Linogliride | 75358-37-1 | U.S. Pat. No. 4211867 |
| | | Issued: Jul. 08, 1980 |
| LY 335563 | 318295-61-3 | WO 2001/026651 |
| | | Issued: Apr. 19, 2001 |
| LY 389382 | 227799-37-3 | EP 924209 |
| | | Issued: Jun. 23, 1999 |
| MCC 555 | 161600-01-7 | U.S. Pat. No. 5594016 |
| | | Issued: Jan. 14, 1997 |
| Ro 16-8714 | 90505-66-1 | EP 101069 |
| | | Issued: Feb. 22, 1984 |
| S 21663 | 162510-01-2 | EP 638568 |
| | | Issued: Feb. 15, 1995 |
| SG 210; | 143162-65-6 | EP 492667 |
| SPR 210 | | Issued: Jul. 01, 1992 |
| SU 4165 | 186371-06-2 | CA 2192796 |
| | | Issued: Dec. 08, 1996 |
| SU 4383 | 186371-07-3 | WO 98/27092 |
| | | Issued: Jun. 25, 1998 |
| SU 4384 | 186371-08-4 | WO 98/27092 |
| | | Issued: Jun. 25, 1998 |
| SU 4386 | 186371-09-5 | WO 98/56376 |
| | | Issued: Dec. 17, 1998 |
| SU 4387 | 186371-10-8 | U.S. Pat. No. 5883110 |
| | | Issued: Mar. 16, 1999 |
| SU 4388 | 186371-11-9 | U.S. Pat. No. 5883110 |
| | | Issued: Mar. 16, 1999 |
| SU 4390 | 186371-12-0 | U.S. Pat. No. 5883110 |
| | | Issued: Mar. 16, 1999 |
| SU 4391 | 186371-13-1 | U.S. Pat. No. 5883110 |
| | | Issued: Mar. 16, 1999 |
| SU 4762 | 186371-14-2 | U.S. Pat. No. 5883110 |
| | | Issued: Mar. 16, 1999 |
| T 1095 | 209746-59-8 | EP 850948 |
| | | Issued: Jul. 01, 1998 |
| T 1095A | 209746-56-5 | JP 2000080041 |
| | | Issued: Mar. 21, 2000 |
| T 0901317 | 293754-55-9 | WO 2000/054759 |
| | | Issued: Sep. 21, 2000 |
| WAY 120744 | 189233-69-0 | WO 98/05331 |
| | | Issued: Feb. 12, 1998 |
| WAY-TES 424 | 198481-33-3 | EP 802183 |
| | | Issued: Oct. 22, 1997 |
| AD 5075 | 103788-05-2 | WO 86/02073 |
| | | Issued: Apr. 10, 1986 |
| AD 5467 | 112808-22-7 | EP 243018 |
| | | Issued: Oct. 28, 1987 |
| BM 131246 | 103787-97-9 | J. Med. Chem. (1992), |
| | | Vol. 35(14), pages |
| | | 2617-2626 |
| Camiglibose | 127214-23-7 | EP 344383 |
| | | Issued: Dec. 06, 1989 |
| JTT 608 | 195137-72-5 | J. Med. Chem. (1998), |
| | | Vol. 41(27), pages |
| | | 5420-5428 |
| KRP 297 | 213252-19-8 | Bioorg. Med. Chem. |
| | | Lett. (1999), Vol. 9(4), |
| | | pages 533-538 |
| LY 275585 | 133107-64-9 | EP 383472 |
| | | Issued: Aug. 22, 1990 |
| M 16209 | 128851-36-5 | EP 355827 |
| | | Issued: Feb. 28, 1990 |
| MDL 25637 | 104343-33-1 | J. Org. Chem. (1989), |
| | | Vol. 54(11), pages |
| | | 2539-2542 |
| Pyrazinoyl- | 60398-24-5 | J. Membr. Biol. (1985), |
| guanidine | | Vol. 83(1-2), |
| | | pages 45-56 |
| RX 871024 | 142872-83-1 | WO 92/06972 |
| | | Issued: Apr. 30, 1992 |
| S 22068 | 162510-35-2 | EP 638568 |
| | | Issued: Feb. 15, 1995 |
| Tolrestat | 82964-04-3 | EP 59596 |
| | | Issued: Sep. 08, 1982 |
| SAH 51-641 | 91456-99-4 | GB 2202849 |
| | | Issued: Oct. 05, 1988 |
| TZD 300512 | 103926-56-3 | J. Med. Chem. (1992), |
| | | Vol. 35(14), pages |
| | | 2617-2726 |
| WAG 994 | 130714-47-5 | Synth. Commun. (1996), |
| | | Vol. 26(21), pages |
| | | 3967-3977 |
| YM 268 | 141716-96-3 | WO 92/00967 |
| | | Issued: Jan. 23, 1992 |
| ZD 4522 | 147098-20-2 | EP 521471 |
| | | Issued: Jan. 07, 1993 |
| FK-614 | insulin sensitizer | Diabetes 2001, 50: |
| | | Suppl 6 (Abs 2180-PO) |
| EML-16257 | glucose-dependent | |
| | beta cell sensitizer | |
| | and insulin | |
| | secretagogue | |
| EML-4156 | insulin sensitizer | |
| EML-16336 | insulin sensitizer | |
| AD-9677 | beta3 adrenergic | |
| | agonist | |
| AZ-40140/ | beta3 adrenergic | |
| SB-418790 | agonist | |
| CLX-0901 | insulin sensitizer | |
| CLX-0921 | PPARgamma agonist | |
| R-483 | PPARgamma agonist | |
| Netoglitazone | PPARgamma agonist | |
| AZ242/ | PPARgamma agonist | |
| tesaglitazar/ | | |
| Galida | | |
| NN-2344/ | PPAR agonist | |
| balaglitazone | | |
| BMS-298585 | PPARalpha/gamma | |
| | agonist | |
| Dexlipotam | enantiomer of | |
| | alpha-lipoic acid: | |
| | for diabetic | |
| | complications and | |
| | possibly glucose | |
| | lowering | |
| NCX-4016 | a nitric oxide- | |
| | releasing non- | |
| | steroidal anti- | |
| | inflammatory drug | |
| | (NO-NSAID) that | |
| | inhibits cyclooxygenase | |
| Telik's | multiple compounds | |
| insulin | | |
| receptor | | |
| activators | | |
| ISIS-113715 | Antisense inhibitor | |
| | of PTP-1B | |
| Exubera/ | Inhaled insulin | |
| HMR-4006 | | |
| AIR (insulin) | Inhaled insulin | |
| Spiros insulin) | Inhaled insulin | |
| AeroDose/ | Inhaled insulin | |
| AeroGen insulin | | |
| AERx insulin | Inhaled insulin | |
| Macrosol | Inhaled insulin | |
| (insulin) | | |
| GW-843362/ | Oral insulin | |
| M2/HIM2 | | |
| Oralin/Oralgen/ | Oral insulin | |
| 9004-10-8 | | |
| Eligen/oral | Oral insulin | |
| insulin(CADDYS) | | |
| L783, 281/ | Insulin receptor | Science (1999), Vol. |
| 78860-34-1/ | activator | 284, pages 974-977 |
| Compound 1 | | |
| Compound 2 | Insulin receptor | J. Biol. Chem. (2000), |
| | activator | Vol. 275(47), pages |
| | | 36590-36595 |
| BVT.2733 | 11-beta- | Diabetologia (2002), |
| | hydroxysteroid | Vol. 45, pages |
| | dehydrogenase-1 | 1528-1532 |
| | (11-beta-HSD1) | |
| | inhibitors | |
| Skyrin/ | Glucagon receptor | |
| rhodophyscin/ | antagonist | |
| endothianin/ | | |
| 606-06-2 | | |
| CP-99711/ | Glucagon receptor | |
| 149839-55-4/ | antagonist | |
| 149366-39-2 | | |
| NNC-25-2504 | Glucagon receptor | J. Med. Chem. (2002), |
| | antagonist | Vol. 45(26), pages |
| | | 5755-5775 |
| BAY-27-9955 | Glucagon receptor | |
| | antagonist | |
| L-168049 | Glucagon receptor | |
| | antagonist | |
| desPhe(6), | Glucagon receptor | |
| Glu(9)glucagons | antagonist | |
| amide | | |
| CP-472555 | Glucocorticoid | EP 1097709, WO 0066522 |
| | antagonists | |
| A-216054 | Glucocorticoid | |
| | antagonists | |
| GP-3034/CS-917/ | Purine nucleotide | |
| MB-6322 | analog and | |
| | fructose-1,6- | |
| | bisphosphatase | |
| | inhibitor | |
| Somatokine/ | rhIGF-1 combined with | |
| rhIGF-BP3/ | IGF-binding protein-3 | |
| IGF-1-BP3 | | |
| fusion protein | | |
| Acetyl CoA | | |
| Carboxylase | | |
| Inhibitors | | |
| CT-98023, | Glycogen Synthase | |
| CT-98014, | Kinase-3 inhibitors | |
| CT-20026 and | | |
| related compounds | | |
| NNC-57-0511, | Glycogen Synthase | |
| NNC-57-0545, | Kinase-3 inhibitors | |
| NNC-57-0588 and | | |
| related compounds | | |
| SB-495052, | Glycogen Synthase | |
| SB-517955, | Kinase-3 inhibitors | |
| SB-410111 and | | |
| related compounds | | |
| GDF-8 program, | Antibody-mediated | |
| anti-myostatin | blockade of | |
| antibody, MYO-029 | myostatin action | |
| LY-333531/ | Protein Kinase C | |
| ruboxistaurin | inhibitors | |
| ALT-946 | Inhibitor of | |
| | Advanced Glycosylation | |
| | Endproduct formation | |
| ALT-711/N- | Advanced Glycosylation | |
| phenacylthiazolium | Endproduct (AGE) | |
| bromide/PTB | breaker | |
| TRC-41XX | Advanced Glycosylation | |
| | Endproduct (AGE) | |
| | breaker | |
| OPB-9195 | Advanced Glycosylation | |
| | Endproduct (AGE) | |
| | breaker | |
| KRX-101/ | Medium molecular weight | |
| Sulodexide | glycosaminoglycans | |

A further embodiment includes products of Table 5.

TABLE 5

| Product | |
|---|---|
| Actos | PPAR-gamma agonists |
| Amaryl | sulfonylureas |
| Avandia | PPAR-gamma agonists |
| Diabeta | sulfonylureas |
| Glucophage | oral hypoglycemic agent |
| Glucophage XR | oral hypoglycemic agent |
| Glucotrol | sulfonylureas |
| Glucovance | metformin combined the |
| | sulfonylurea, glyburide |
| Glynase PresTab | sulfonylureas |
| Glyset | sulfonylureas |
| Micronase | sulfonylureas |
| Prandin | glitinides |
| Precose | oral hypoglycemic agent |
| Starlix | glitinides |
| Humalog | Insulin |
| Humalog 50/50 | Insulin |
| Humalog 75/25 | Insulin |
| Humulin 50/50 | Insulin |
| Humulin 75/25 | Insulin |
| Humulin L | Insulin |
| Humulin N | Insulin |
| Humulin R | Insulin |
| Humulin R U-500 | Insulin |
| HumulinU | Insulin |
| Iletin II Lente | Insulin |
| Iletin II NPH | Insulin |
| Iletin II Regular | Insulin |
| Lantus | Insulin |
| Novolin L | Insulin |
| Novolin N | Insulin |
| Novolin R | Insulin |
| Novolog | Insulin |
| Velosulin BR | Insulin |

A further embodiment includes dipeptidyl peptidase IV (DPP-IV) inhibitors of Tables 6 and 7.

TABLE 6

| Generic Name(s) | CAS* Registry Number | | |
|---|---|---|---|
| of DPP-IV | and | | Reference to Source of |
| Inhibitor | Chemical Name | Chemical Structure | Compound |
| | 133746-77-7; Benzoic acid, 4-[[1-[4-(1,1- dimethylethyl) phenyl]-5-oxo-3- pyrrolidinyl]methoxy]- | 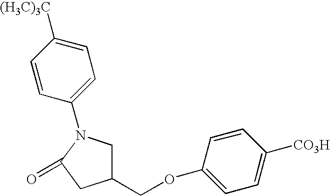 | European Patent Application EP 393607 Date of Publication: Oct. 24, 1990 |
| | 155730-92-0; Benzoic acid, 4-[[(3S)-1-[4-(1,1- dimethylethyl) phenyl]-5-oxo-3- pyrrolidinyl]methoxy]- | 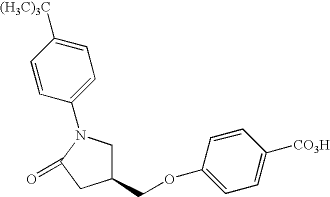 | European Patent Application EP 393607 Date of Publication: Oct. 24, 1990 |
| O-Benzoyl hydroxyamine | 54495-98-6; Hydroxylamine, O-benzyl- | 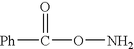 | Synthesis (1975), (12), 788-9. |
| Diprotin A | 90614-48-5; L-Isoleucine, L-isoleucyl-L-prolyl- | 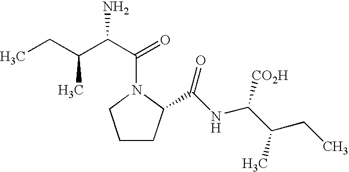 | Japanese Patent JP 59025366 Date of Issue: Feb. 9, 1984 |
| Diprotin B | 90614-49-6; L-Leucine, L-valyl-L-prolyl- | 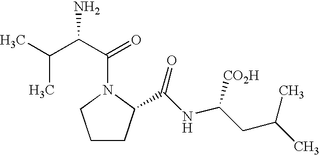 | Japanese Patent JP 59025366 Date of Issue: Feb. 9, 1984 |
| Diprotin C | 90632-50-1; L-Leucine, L-valyl-L-prolyl- | 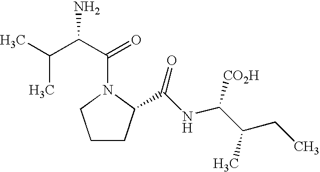 | Japanese Patent JP 59025366 Date of Issue: Feb. 9, 1984 |
| FE 999011 | 171092-64-1; 2-Pyrrolidinecarbonitrile, 1-[(2S)-2- amino-3,3-dimethyl-1-oxobutyl]-,(2S)- | 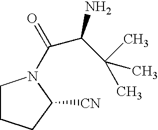 | PCT Patent Appli- cation WO 9515309 Date of Publication: Jun. 8, 1995 |
| NVP-DPP 728 | 247016-69-9; 2-Pyrrolidinecarbonitrile, 1-[[[2-[(5- cyano-2- pyridinyl)amino]ethyl]amino]acetyl]-, (2S)- | 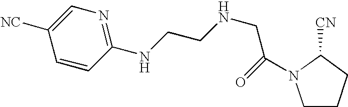 | U.S. Patent 6011155 Date of Issue: Jan. 4, 2000 |
| P32/98 | 251572-86-8; Thiazolidine, 3-[(2S, 3S)-2-amino-3- methyl-1-oxopentyl]-,(2E)-2- Butenedioate (2:1) | 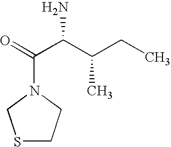 Difumarate Salt | Drugs of the Future (2001), 26(9), 859-864. |
| L-Proline, 1-L- valyl-; Proline, 1-L-valyl-, L- (8Cl); Proline, 1-valyl- (6Cl); L-Valine-L proline; L-Valyl-L- proline; N-Valyl-L- proline | 20488-27-1; L-Proline, L-valyl- | 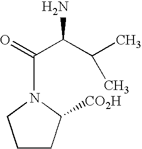 | Zhurnal Obshchei Khimii (1990), 60(1), 170-5. |
| Pyrrolidine, 1-(2- amino-1- oxopropyl)-, (S)-; L-Alanyl pyrrolidine | 56384-04-4; Pyrrolidine, 1-[(2S)-2-amino-1- oxopropyl]- | 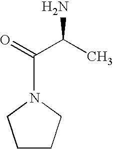 | Bioorganic & Medicinal Chemistry Letters (1996), 6(10), 1163-1166. |
| SDZ 029-576 | 294619-41-3; 4-Isoquinolineacetic acid, 1- (aminomethyl)-6,7-dimethoxy-, ethyl ester, dihydrochloride | 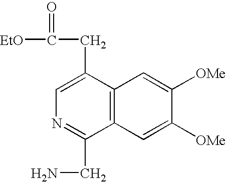 | Biorganic and Medicinal Chemistry Letters (2000), 10(14), 1555-1558. |
| Sulfostin | 307345-51-3; Sulfamic acid, [(R)-amino[(3S)-3- amino-2-oxo-1- piperidinyl]phosphinyl]- | 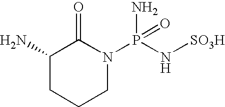 | Journal of Antibiotics (2001), 54(9), 744-746. |
| TMC 2A | 195976-77-3; L-Leucme, N-[[(3S)-2-[(2S)-2-amino- 3-(1H-indol-3-yl)-1-oxopropyl]- 1,2,3,4-tetrahydro-6,8-dihydroxy-7- methoxy-3-isoquinolinyl]carbonyl]- 5,5′-dihydroxy- | 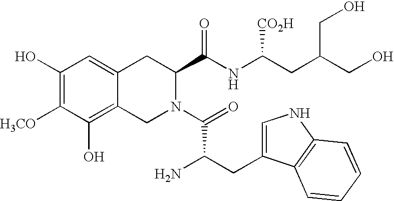 | Journal of Antibiotics (1997), 50(8), 653-658. |
| TMC 2B | 196212-07-4; L-Leucine, L-tryptophyl-(3S)-1,2,3,4- tetrahydro-6,8-dihydroxy-7-methoxy-3- isoquinolinecarbonyl-5-hydroxy- | 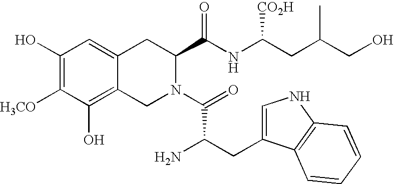 | Journal of Antibiotics (1997), 50(8), 653-658. |
| TMC 2C | 196212-08-5; L-Leucine, L-tryptophyl-(3S)-1,2,3,4- tetrahydro-6,8-dihydroxy-7-methoxy-3- isoquinolinecarbonyl-5-hydroxy- | 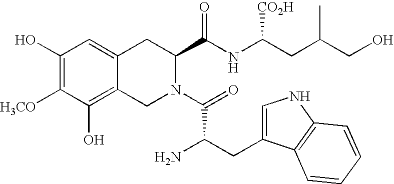 | Journal of Antibiotics (1997), 50(8), 653-658. |
| TSL-225 | 211169-95-8; 3-Isoquinolinecarboxylic acid, 2-[(2S)- 2-amino-3-(1H-indol-3-yl)-1- oxopropyl]-1,2,3,4-tetrahydro-,(3S)- | 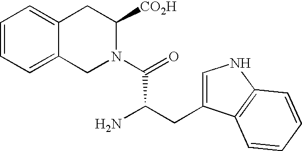 | Bioorganic & Medicinal Chemistry Letters (1998), 8(12), 1537-1540. |

TABLE 7

| | | Reference to Source of |
|---|---|---|
| Company | Chemical Type | Inhibitor Compounds of DPP-IV |
| Les | Alpha-amino Acid Derivatives | European Patent |
| Laboratoires | | Application 1258476 |
| Servier | | Date of Publication: |
| | | Nov. 20, 2002 |
| Bristol-Myers | 2,1-Oxazoline and | PCT Int. Appl. |
| Squibb | 1,2-Pyrazoline-Based | WO 2002083128 |
| | Inhibitors | Published: |
| | | Oct. 24, 2002 |
| Merck | Carbonyl Derivatives of | PCT Int. Appl. |
| | Thiazolidine | WO 2002076450 |
| | | Published: |
| | | Oct. 3, 2002 |
| Les | Amino Acid Sulfonyl Derivatives | European Patent |
| Laboratoires | | Application 1245568 |
| Servier | | Date of Publication: |
| | | Oct. 2, 2002 |
| Mitsubishi | N-(α-Aminoacyl)-2- | Japanese Patent 2002265439 |
| Well Pharma | Cyanopyrrolidine Derivatives | Date of Issue: |
| | | Sep. 18, 2002 |
| Boehringer | Xanthine Derivatives | PCT Int. Appl. |
| Ingelheim | | WO 2002068420 |
| | | Date of Publication: |
| | | Sep. 6, 2002 |
| Boehringer | Xanthines | German Patent |
| Ingelheim | | DE 10109021 |
| | | Date of Issue: |
| | | Sep. 5, 2002 |
| Takeda | Isoquinolinones | PCT Int. Appl. |
| Chemical | | WO 2002062764 |
| Industries | | Date of Publication: |
| | | Aug. 15, 2002 |
| Kyowa Hakko | Aminocarbonylpyrrolidine | PCT Int. Appl. |
| Kogyo Co. | Derivatives | WO 2002051836 |
| | | Date of Publication: |
| | | Jul. 4, 2002 |
| Taisho | 2-Cyanopyrrolidine Derivatives | PCT Int. Appl. |
| Pharmaceutical | | WO 2002038541 |
| | | Date of Publication: |
| | | May 16, 2002 |
| Tanabe | Aliphatic Nitrogenous | PCT Int. Appl. |
| Seiyaku | Five-membered Ring Compounds | WO 2002030891 |
| | | Published: |
| | | Apr. 18, 2002 |
| Tanabe | Nitrogenous Five-membered | PCT Int. Appl. |
| Seiyaku | Ring Compounds Such As | WO 2002030890 |
| | (S)—N—[N-Cyclohexyl or | Published: |
| | N-(4-Piperidinyl)glycyl]pyrrolidine- | Apr. 18, 2002 |
| | 2-Carbonitrile | |
| Ilex Oncology | α-Substituted | PCT Int. Appl. |
| Research | β-Aminoethyl Phosphonates | WO 2002026752 |
| | | Published: |
| | | Apr. 4, 2002 |
| Welfide | Proline Derivatives | PCT Int. Appl. |
| Corporation | | WO 2002014271 |
| | | Date of Publication: |
| | | Feb. 21, 2002 |
| Novo Nordisk | Piperazinylpurinediones | PCT Int. Appl. |
| A/S | | WO 2002002560 |
| | | Date of Publication: |
| | | Jan. 10, 2002 |
| Novartis AG | N-Glycyl-2-Cyanopyrrolidines | PCT Int. Appl. |
| | | WO 2001096295 |
| | | Date of Publication: |
| | | Dec. 20, 2001 |
| Ferring Bv | Peptidomimetics | PCT Int. Appl. |
| | | WO 2001081337 |
| | | Date of Publication: |
| | | Nov. 1, 2001 |
| Ferring Bv | Peptidomimetics | PCT Int. Appl. |
| | | WO 2001081304 |
| | | Date of Publication: |
| | | Nov. 1, 2001 |
| Bristol-Myers | Fused Cyclopropylpyrrolidine- | PCT Int. Appl. |
| Squibb | Based Inhibitors | WO 2001068603 |
| | | Date of Publication: |
| | | Sep. 20, 2001 |
| Novo Nordisk | N-Aminoalkanoylpyrroli(di)ne-2- | PCT Int. Appl. |
| A/S | Carbonitriles | WO 2001055105 |
| | | Date of Publication: |
| | | Aug. 2, 2001 |
| Ferring Bv | 1-(2′-Aminoacyl)-2- | PCT Int. Appl. |
| | Cyanopyrrolidine Derivatives | WO 2001040180 |
| | | Date of Publication: |
| | | Jun. 7, 2001 |
| Probiodrug | Peptide Derivatives | PCT Int. Appl. |
| Gesellschaft for | | WO 2001014318 |
| Arzneimittle- | | Published: |
| forschung | | Mar. 1, 2001 |
| Novartis AG | Tetrahydroisoquinoline-3- | U.S. Pat. No. 6172081 |
| | Carboxamide Derivatives | Date of Issue: |
| | | Jan. 9, 2001 |
| Zaidan Hojin | Sulfostin Analogues | PCT Int. Appl. |
| Biseibutsu | | WO 2000069868 |
| Kagaku Kenkyu | | Date of Publication: |
| Kai | | Nov. 23, 2000 |
| Novartis AG | 3-[(Alkylamino)acetyl]-4- | U.S. Pat. No. 6110949 |
| | Cyanothiazolidines | Date of Issue: |
| | | Aug. 29, 2000 |
| Novartis AG | 1-Aminomethylisoquinoline-4- | Biorganic and Medicinal |
| | carboxylates | Chemistry Letters (2000), |
| | | 10(14), 1555-1558. |
| Novartis AG | N-Glycyl-2-Cyanopyrrolidines | PCT Int. Appl. |
| | | WO 2000034241 |
| | | Date of Publication: |
| | | Jun. 15, 2000 |
| Novartis AG | Aminoacetylthiazolidines | U.S. Pat. No. 6107317 |
| | | Date of Issue: |
| | | Aug. 22, 2000 |
| Novartis AG | N-(Substituted Glycyl)-2- | U.S. Pat. No. 6011155 |
| | Cyanopyrrolidines | Date of Issue: |
| | | Jan. 4, 2000 |
| Martin-Luther- | Thioxo Amino Acid Pyrrolidides | Biochimica et Biophysica |
| Universitat | and Thiazolidides | Acta (2000), 1479(1-2), |
| Halle-Wittenberg | | 15-31. |
| Probiodrug | Prodrugs of DPP-IV Inhibitors | PCT Int. Appl. |
| Gesellschaft fur | | WO 9967279 |
| Arzneimittle- | | Date of Publication: |
| forschung | | Dec. 29, 1999 |
| Probiodrug | Prodrugs of DPP-IV Inhibitors | PCT Int. Appl. |
| Gesellschaft fur | | WO 9967278 |
| Arzneimittle- | | Date of Publication: |
| forschung | | Dec. 29, 1999 |
| Probiodrug | New DPP-IV Effectors | PCT Int. Appl. |
| Gesellschaft fur | | WO 9961431 |
| Arzneimittle- | | Date of Publication: |
| forschung | | Dec. 2, 1999 |
| Taiho | Phenylcarboxylic Acid Derivatives | PCT Int. Appl. |
| Pharmaceutical | | WO 9943318 |
| Co. | | Date of Publication: |
| | | Sep. 2, 1999 |
| University of | Diaryl Phosphonate Esters | Journal of Medicinal |
| Antwerp | | Chemistry (1999), 42(6), |
| | | 1041-1052. |
| State University | Fluoroolefin-Containing | Proceedings of the |
| of New York at | N-Peptidyl-O-Hydroxylamine | U.S. National Academy |
| Albany | Peptidomimetics | of Sciences (1998), |
| | | 95(24), 14020-14024. |
| Institute of | Sulfostin | Journal of Antibiotics |
| Microbial | | (2001), 54(9), 744-746. |
| Chemistry, Tokyo | | |
| University of | Diaryl Phosphonate Esters | Proceedings of the 25^{th} |
| Antwerp | | European Peptide Symposium, |
| | | Budapest, Aug. 30-Sep. |
| | | 4, 1998 (1999), 818-819. |
| University of | N-Phenylphthalimide Analogs | Bioorganic & Medicinal |
| Tokyo | | Chemistry Letters (1999), |
| | | 9(4),559-562. |
| Tanabe Seiyaku | Dipeptide Inhibitor | Bioorganic & Medicinal |
| Co. | | Chemistry Letters (1998), |
| | | 8(12), 1537-1540. |
| Universite de | Cyclopeptide Inhibitors | Journal of Medicinal |
| Versailles | | Chemistry (1998), 41(12), |
| | | 2100-2110. |
| Novartis AG | N-Aminoacetyl-2- | PCT Int. Appl. |
| | Cyanopyrrolidines | WO 9819998 |
| | | Date of Publication: |
| | | May 14, 1998 |
| Tanabe Seiyaku | Amino Acid-containing | PCT Int. Appl. |
| | Tetrahydroquinoline Derivatives | WO 9818763 |
| | | Date of Publication: |
| | | May 7, 1998 |
| Nippon Shinyaku | Carboxylic Acid Derivatives | PCT Int. Appl. |
| Co. | | WO 9715546 |
| | | Date of Publication: |
| | | May 1, 1997 |
| Warner-Lambert | Sulfamic Acid Derivatives, | PCT Int. Appl. |
| | Acyl Sulfonamides or Sulfonyl | WO 9705868 |
| | Carbamates | Date of Publication: |
| | | Feb. 20, 1997 |
| Symphar S.A.; | Aminophosphonates | PCT Int. Appl. |
| Smithkline | α-Substituted by Phenol | WO 9702037 |
| Beecham | Groups | Date of Publication: |
| | | Jan. 23, 1997 |
| Lead Generation | TMC-2A, 2B, and -2C | Journal of Antibiotics |
| Research Labora- | | (1997), 50(8), 653-658. |
| tory, Toda, Japan | | |
| University of | Pyrrolidides | European Journal of |
| Antwerp | | Medicinal Chemistry (1997), |
| | | 32(4), 301-309. |
| Ferring Research | 4-Cyanothiazolidides | Bioorganic & Medicinal |
| Institute | | Chemistry Letters (1996), |
| | | 6(22), 2745-2748. |
| Ferring Research | 4-Cyanopyrrolidides | Bioorganic & Medicinal |
| Institute | | Chemistry Letters (1996), |
| | | 6(10), 1163-1166. |
| Boehringer | Boronic Acid Inhibitors | Journal of Medicinal |
| Ingelheim | | Chemistry (1996), 39(10), |
| Pharmaceutical | | 2087-2094. |
| State University | Fluorolefin Isosteres | ACS Symposium Series |
| of New York - | | (1996), 639, 129-142. |
| Albany | | |
| State University | Fluorolefin Containing | Tetrahedron (1996), |
| of New York - | Dipeptide Isosteres | 52(1), 291-304. |
| Albany | | |
| Georgia Tech. | Peptide Containing Proline | PCT Patent Application |
| Research Corp. | Phosphonate Derivatives | WO 9529691 |
| | | Date of Publication: |
| | | Nov. 9, 1995 |
| Ferring B.V. | Peptide Analog DP-IV Serine | PCT Int. Appl. |
| | Protease Inhibitors | WO 9515309 |
| | | Date of Publication: |
| | | Jun. 8, 1995 |
| University of | Azaproline Peptides | Letters in Peptide |
| Antwerp | | Science (1995), |
| | | 2(3/4), 198-202. |
| Mount Sinai | Aminoacylpyrrolidine-2-nitriles | Archives of Biochemistry |
| School of | | and Biophysics (1995), |
| Medicine | | 323(1), 148-154. |
| Georgia Tech. | Dipeptide Phosphonates | Journal of Medicinal |
| Research | | Chemistry (1994), 37(23), |
| Corp. | | 3969-3976. |
| Taiho | Optically Active 1- | PCT Patent Application |
| Pharmaceutical | Phenylpyrrolidone Derivatives | WO 9406767 |
| | | Date of Publication: |
| | | Mar. 31, 1994 |
| New England | Peptidylboronate Derivatives | PCT Patent Application |
| Medical Center | | WO 9308259 |
| Hospitals; Tufts | | Date of Publication: |
| University | | Apr. 29, 1993 |
| Otsuka Seiyaku | (Piperidinyalkoxy- or | Japanese Patent |
| | Pyrrolidinylalkoxy)benzoic | JP 04112868 |
| | Acid Derivatives | Date of Issued: |
| | | Apr. 14, 1992 |
| Martin-Luther- | Amino Acid Amides | East German Patent |
| Universitaet | | DD 296075 |
| Halle-Wittenberg | | Date of Issued: |
| | | Nov. 21, 1991 |
| Otsuka | 4-[1-(Substituted)phenyl-2- | European Patent |
| Pharmaceutical | Pyrrolidon-4-yl]methoxybenzoic | Application EP 393607 |
| Co. | Acids and Analogs | Date of Publication: |
| | | Oct. 24, 1990 |
| Martin Luther | N-peptidyl-O- | Journal of Organic |
| Univ., Halle/ | (nitrobenzoyl)hydroxylamines | Chemistry (1989), 54(25), |
| Saale | | 5880-5883. |

Another embodiment includes protein tyrosine phosphatase 1B (PTP 1B) inhibitors of Table 8.

TABLE 8

| | | Reference to Source of |
|---|---|---|
| Company | Chemical Type | Inhibitor Compounds of PTP 1B |
| Chinese | Natural PTP 1B Inhibitors | Bioorganic and Medicinal |
| Academy of | | Chemistry Letters (2002 |
| Sciences | | Dec.), 12(23), 3387-3390. |
| Abbott | Amino(oxo)acetic Acid Derivatives | U.S. Pat. Appl. |
| Laboratories | | U.S. Pat. No. 20020169157 |
| | | Published: |
| | | Nov. 14, 2002 |
| | Phenylalkanone Oximes | Japanese Patent 2002322141 |
| | | Published: |
| | | Nov. 8, 2002 |
| Brown | Divalent and Trivalent | Journal of Medicinal |
| University | α-Ketocarboxylic Acids | Chemistry (2002), |
| | | 45(18), 3946-3952. |
| Merck | 2-Aryloxy-2-Arylalkanoic Acids | PCT Int. Appl. |
| | | WO 2002064094 |
| | | Published: |
| | | Aug. 22, 2002 |
| Korean | 1,2-Naphthoquinone Derivatives | Bioorganic and Medicinal |
| Research | | Chemistry Letters (2002 |
| Institute | | Aug. 5), 12(15), |
| | | 1941-1946. |
| | Substituted Phenylalaninol | U.S. Pat. No. 6,410,585 |
| | Derivatives | Date of Patent: |
| | | Jun. 25, 2002 |
| Abbott | Dichlorophenoxy(benzyl)acetic | U.S. Pat. Appl. |
| Laboratories | Acid Derivatives | U.S. Pat. No. 2002077347 |
| | | Date of Publication: |
| | | Jun. 20, 2002 |
| Abbott | Amino(oxo)acetic Acids | U.S. Pat. Appl. |
| Laboratories | | U.S. Pat. No. 2002072516 |
| | | Date of Publication: |
| | | Jun. 13, 2002 |
| Biovitrum AB | Tetrazole-Containing Peptidomimetic | Journal of Medicinal |
| | Inhibitors | Chemistry (2002), |
| | | 45(9), 1785-1798. |
| Japan Tobacco | 2-(2,5-Dihalo-3,4- | Japanese Patent 2002114768 |
| | Dihydroxyphenyl)azole Derivatives | Date of Issue: |
| | | Apr. 16, 2002 |
| Abbott | Amino(oxo)acetic Acid Derivatives | U.S. Pat. Appl. |
| Laboratories | | U.S. Pat. No. 2002035136 |
| | | Date of Publication: |
| | | Mar. 21, 2002 |
| Abbott | Aryloxybenzylacetic Acids | PCT Int. Appl. |
| Laboratories | | WO 2002018363 |
| | | Published: |
| | | Mar. 7, 2002 |
| Abbott | Amino(oxo)acetic Acids | PCT Int. Appl. |
| Laboratories | | WO 2002018321 |
| | | Published: |
| | | Mar. 7, 2002 |
| Abbott | Amino(oxo)acetic Acids | PCT Int. Appl. |
| Laboratories | | WO 2002018323 |
| | | Date of Publication: |
| | | Mar. 7, 2002 |
| Pharmacia | Peptidomimetic Competitive | Journal of Medicinal |
| | Inhibitors | Chemistry (2002), |
| | | 45(3), 598-622. |
| Aventis Pharma | Substituted and Non-Substituted | PCT Int. Appl. |
| Deutschland | Benzooxathiazoles | WO 2002011722 |
| | | Date of Publication: |
| | | Feb. 14, 2002 |
| Array Biopharma | α-Arylsulfonylamino-α- | PCT Int. Appl. |
| | Benzylcarboxamides | WO 2002004412 |
| | | Published: |
| | | Jan. 17, 2002 |
| Novo Nordisk; | Thienopyridines | PCT Int. Appl. |
| Ontogen Corp. | | WO 2002004458 |
| | | Date of Publication: |
| | | Jan. 17, 2002 |
| Novo Nordisk; | 2-Oxalylaminothieno[2,3- | PCT Int. Appl. |
| Ontogen Corp. | c]pyridines | WO 2002004459 |
| | | Date of Publication: |
| | | Jan. 17, 2002 |
| Takeda Chemical | Pyrrole Derivatives | PCT Int. Appl. |
| Industries | | WO 2001090067 |
| | | Date of Publication: |
| | | November 29, 2001 |
| Takeda Chemical | Bis-indolyl Benzoquinone | Japanese Patent Appl. |
| Industries | | JP 2001302629 |
| | | Published: |
| | | Oct. 31, 2001 |
| University of | Quinolinedione | Journal of Medicinal |
| Pittsburgh | | Chemistry, (2001), |
| | | 44(24), 4042-4049 |
| Merck Frosst | Sulfur Substituted | PCT Int. Appl. |
| Canada; Banyu | Naphthyldifluoromethylphosphonic | WO 2001070754 |
| Pharmaceutical | Acids | Published: |
| | | Sep. 27, 2001 |
| Merck Frosst | Sulfur Substituted | PCT Int. Appl. |
| Canada | Phenyldifluoromethylphosphonic Acids | WO 2001070753 |
| | | Published: |
| | | Sep. 27, 2001 |
| American Home | (2-Acylaminothiazol-4-yl)acetic | U.S. Pat. No. 6281234 |
| Products | Acid Derivatives | Date Issued: |
| | | Aug. 28, 2001 |
| Merck Frosst | Phosphonic Acid Biaryl Derivatives | PCT Int. Appl. |
| Canada | | WO 2001046203 |
| | | Date of Publication; |
| | | Jun. 28, 2001 |
| Merck Frosst | Aromatic Phosphonates | PCT Int. Appl. |
| Canada | | WO 2001046204 |
| | | Date of Publication; |
| | | Jun. 28, 2001 |
| Merck Frosst | Phosphonic Acid Derivatives | PCT Int. Appl. |
| Canada | | WO 2001046205 |
| | | Date of Publication; |
| | | Jun. 28, 2001 |
| Merck Frosst | Phosphonic Acid Derivatives | PCT Int. Appl. |
| Canada | | WO 2001046206 |
| | | Published: |
| | | Jun. 28, 2001 |
| American Home | Benzothiophenes, Benzofurans, | U.S. Pat. No. 6251936 |
| Products | and Indoles | Date of Issue: |
| | | Jun. 26, 2001 |
| American Home | α-(Biphenylyloxo)alkanoic Acids | U.S. Pat. No. 6232322 |
| Products | | Date of Issue: |
| | | May 15, 2001 |
| American Home | [[(Benzofuranylbiphenylyl)oxy]- | U.S. Pat. No. 6221902 |
| Products | sulfonyl]benzoates and Analogs | Date of Issue: |
| | | Apr. 24, 2001 |
| Pharmacia | Small Molecule Peptidomimetics | Biochemistry (2001), |
| | | 40(19), 5642-5654. |
| Astra Zeneca | 9,10-Phenanthrenedione Inhibitors | Journal of Medicinal |
| Pharmaceutical | | Chemistry (2001), |
| | | 44(11), 1777-1793. |
| Novo Nordisk A/S | 2-Amino-4H-thiazolo[5,4-b]indole | Journal of Heterocyclic |
| | Conversion Products | Chemistry (2001), 38(3), |
| | | 569-577. |
| | Bi- and Terphenylcarboxamides | U.S. Pat. No. 6214877 |
| | | Date of Issue: |
| | | Apr. 10, 2001 |
| Novo Nordisk; | 2-(Oxalylamino)-4,5,6,7- | PCT Int. Appl. |
| Ontogen Corp. | Tetrahydrothieno[2,3-c]pyridine- | WO 2001019830 |
| | 3-carboxylic Acids | Date of Publication: |
| | | Mar. 22, 2001 |
| Novo Nordisk; | 2-(Oxalylamino)-4,7-Dihydro-5H- | PCT Int. Appl. |
| Ontogen Corp. | Thieno[2,3-c]pyran-3-carboxylic | WO 2001019831 |
| | Acids | Date of Publication: |
| | | Mar. 22, 2001 |
| Sugen, Inc. | Aromatic Trifluoromethylsulfonyl and | PCT Int. Appl. |
| | Trifluoromethylsulfonamido Compounds | WO 2001016097 |
| | | Date of Publication: |
| | | Mar. 8, 2001 |
| University of | Sulfonylated Aminothiazoles | Bioorganic & Medicinal |
| Pittsburgh | | Chemistry Letters (2001), |
| | | 11(3), 313-317 |
| Taisho | 2-{[4-(Methylthio)pyridin-2- | Bioorganic & Medicinal |
| Pharmaceutical | yl]methylsulfinyl}benzimidazole | Chemistry Letters (2000), |
| | | 10(23), 2657-2660. |
| Merck Frosst | Phosphonic and Carboxylic Acid | PCT Int. Appl. |
| Canada | Derivatives | WO 2000069889 |
| | | Date of Publication: |
| | | November 23, 2000 |
| American Home | 11-Aryl-benzo[b]naphtho[2,3- | U.S. Pat. No. 6110962 |
| Products | d]furans and 11-Aryl- | Date of Issue: |
| | benzo[b]naphtho[2,3- | August 29, 2000 |
| | d]thiophenes | |
| Wyeth-Ayerst | 4-Aryl-1-Oxa-9- | Bioorganic & Medicinal |
| Research | Thiacyclopenta[b]fluorenes | Chemistry Letters (2000), |
| | | 10(14), 1535-1538. |
| American Home | 4-Aryloxysulfonyl-2-Hydroxybenzoates | U.S. Pat. No. 6063815 |
| Products | and Analogs | Date of Issue: |
| | | May 16, 2000 |
| Warner-Lambert | 11-Aryl-benzo[b]naphtho[2,3- | Chemtracts (2000), |
| | d]furans and 11-Aryl- | 13(4), 259-264. |
| | benzo[b]naphtho[2,3- | |
| | d]thiophenes | |
| Taiho | Nocardinones A and B | Journal of Antibiotics |
| Pharmaceutical | | (2000), 53(4), 337-344. |
| American Home | 4-Aryl-1-Oxa-9- | U.S. Pat. No. 6057316 |
| Products | Thiacyclopenta[b]fluorenes | Date of Issue: |
| | | May 2, 2000 |
| University of | Chiral ∝-Monofluorophosphonic | Perkin 1 (2000), (8), |
| Toronto | Acids and Derivatives | 1271-1281. |
| Merck Frosst | Phosphonic Acid Derivatives | PCT Int. Appl. |
| Canada | | WO 2000017211 |
| | | Date of Publication: |
| | | Mar. 30, 2000 |
| New York | Non-Peptidyl | Bioorganic & Medicinal |
| University | Aryloxymethylphosphonates | Chemistry Letters (2000), |
| | | 10(5), 457-460. |
| Institute for | 3,4-Dephostatin Derivatives | Tetrahedron (2000), 56(5), |
| Microbial | | 741-752. |
| Chemistry, Tokyo | | |
| Wyeth-Ayerst | Benzofuran and Benzothiophene | Journal of Medicinal |
| Research, Inc. | Biphenyls | Chemistry (2000), |
| | | 43(7), 1293-1310. |
| Wyeth-Ayerst | Azolidinediones | Journal of Medicinal |
| Research, Inc. | | Chemistry (2000), |
| | | 43(5), 995-1010. |
| American Home | 1-Aryldibenzothiophenes | U.S. Pat. No. 6001867 |
| Products | | Date of Issue: |
| | | December 14, 1999 |
| American Home | α-(Biphenylyloxo)alkanoic Acids | PCT Int. Appl. |
| Products | | WO 9958518 |
| | | Date of Publication: |
| | | November 18, 1999 |
| Novo Nordisk; | Bicyclic Heterocyclic Amides | PCT Int. Appl. |
| Ontogen Corp. | | WO 9946268 |
| | | Date of Publication: |
| | | Sep. 16, 1999 |
| Novo Nordisk; | Thieno[2,3-c]pyrans and | PCT Int. Appl. |
| Ontogen Corp. | Thieno[2,3-c]pyridines | WO 9946267 |
| | | Date of Publication: |
| | | Sep. 16, 1999 |
| Novo Nordisk; | Thiophenecarboxylic Acid Derivatives | PCT Int. Appl. |
| Ontogen Corp. | | WO 9946244 |
| | | Date of Publication: |
| | | Sep. 16, 1999 |
| Novo Nordisk; | Oxalylaminothiophene Derivatives | PCT Int. Appl. |
| Ontogen Corp. | | WO 9946237 |
| | | Date of Publication: |
| | | Sep. 16, 1999 |
| Novo Nordisk; | (Oxalylamino)benzoic Acid | PCT Int. Appl. |
| Ontogen Corp. | Derivatives | WO 9946236 |
| | | Date of Publication: |
| | | Sep. 16, 1999 |
| Wyeth-Ayerst | 11-Aryl-benzo[b]naphtho[2,3- | Journal of Medicinal |
| Research, Inc. | d]furans and 11-Aryl- | Chemistry (1999), |
| | benzo[b]naphtho[2,3- | 42(17), 3199-3202. |
| | d]thiophenes | |
| Novo Nordisk; | Thienopyridazinones and | PCT Int. Appl. |
| Ontogen Corp. | Thienochromenones | WO 9915529 |
| | | Date of Publication: |
| | | Apr. 1, 1999 |
| Pharmacia and | Substituted Phenylalanine | PCT Int. Appl. |
| Upjohn Company | Derivatives | WO 9911606 |
| | | Date of Publication: |
| | | Mar. 11, 1999 |
| Yeshiva | bis(Aryldifluorophosponates) | Biochemistry (1999), |
| University | | 38(12), 3793-3803. |
| Merck Frosst | [Difluoro(phosphono)methyl]- | Biochemical Journal |
| Canada | phenylalanine-containing Peptides | (1999), 337(2), |
| | Inhibitors | 219-223. |
| University of | Non-Peptidyl Inhibitors | Bioorganic & Medicinal |
| Toronto | | Chemistry (1998), 6(11), |
| | | 2235. |
| University of | Phosphate Mimetics | Bioorganic & Medicinal |
| Toronto | | Chemistry Letters (1998), |
| | | 8(22), 3275-3280. |
| National | Naphthyldifluoromethylphosphonic | Bioorganic & Medicinal |
| Institutes | Acids | Chemistry (1998), 6(10), |
| of Health | | 1799-1810. |
| University of | α,α-Difluorobenzylphosphonic | Bioorganic & Medicinal |
| Toronto | Acids | Chemistry (1998), 6(9), |
| | | 1457-1468. |
| Merck Frosst | Sulfotyrosyl Peptides | Archives of Biochemistry |
| Canada | | and Biophysics (1998), |
| | | 354(2), 225-231. |
| Ontogen Corp. | (Hetero)arylacrylates | PCT Int. Appl. |
| | | WO 9827065 |
| | | Date of Publication: |
| | | Jun. 25, 1998 |
| University of | Naphthalenebis[α,α- | Bioorganic & Medicinal |
| Toronto | Difluorobenzylphosphonates] | Chemistry Letters (1998), |
| | | 8(4), 345-350. |
| Novo Nordisk | Acrylic Acids | PCT Int. Appl. |
| | | WO 9739748 |
| | | Date of Publication: |
| | | Oct. 30, 1997 |
| Ontogen Corp. | Arylacrylic Acid Derivatives | PCT Int. Appl. |
| | | WO 9708934 |
| | | Date of Publication: |
| | | Mar. 13, 1997 |
| National | Phosphotyrosine-Mimic Containing | Bioorganic & Medicinal |
| Institutes of | Cyclic Peptides | Chemistry (1997), 5(1), |
| Health | | 157-163. |
| National | Difluorophosphonomethyl-containing | Tetrahedron (1996), |
| Institutes of | Phosphatase Inhibitor | 52(30), 9963-9970. |
| Health | | |
| National | Phosphonate Inhibitors | Biochemical Journal (1995), |
| Institute of | | 311(3), 1025-1031. |
| Aging | | |

A further embodiment includes glucagon like peptide-1 (GLP-1) modulators of Table 9.

TABLE 9

| | | Reference to Source |
|---|---|---|
| | | of Modular Compounds |
| Company | Chemical Type or | of GLP-1 |
| Administrators | Cyclic Peptides as | PCT Int. Appl. |
| of the Tulane | Somatostatin Agonists | WO 2002081499 |
| Educational | | Date of Publication: |
| Fund, USA | | Oct. 17, 2002 |
| Amylin | Peptide YY and | PCT Int. Appl. |
| Pharmaceuticals | Peptide YY Agonists | WO 2002047712 |
| | | Date of Publication: |
| | | Jun. 20, 2002 |
| Eli Lilly | GLP-1 Fusion Proteins | PCT Int. Appl. |
| | | WO 2002046227 |
| | | Date of Publication: |
| | | Jun. 13, 2002 |
| General | Vasodilator-Thrombolytic | PCT Int. Appl. |
| Hospital | Fusion Proteins and | WO 2001085100 |
| Corporation | Conjugates | Date of Publication: |
| | | Nov. 15, 2001 |
| Novo Nordisk | Lipophilic Human | U.S. Pat. Appl. Publ. |
| A/S | Glucagon-like Peptide-1 | U.S. Pat. No. 2001011071 |
| | Derivatives | Date of Publication: |
| | | Aug. 2, 2001 |
| Novo Nordisk | Lipophilic Human | U.S. Pat. No. 6,268,343 |
| A/S | Glucagon-like Peptide-1 | Date of Issue: |
| | Derivatives | Jul. 31, 2001 |
| | Protein Homologs | PCT Int. Appl. |
| | | WO 2001053312 |
| | | Date of Publication: |
| | | Jul. 26, 2001 |
| Transkaryotic | Small Peptides from | PCT Int. Appl. |
| Therapies, Inc. | Somatostatin ProPeptide | WO 2001036643 |
| | | Date of Publication: |
| | | May 25, 2001 |
| Novo Nordisk | GLP-1 Agonists, | PCT Int. Appl. |
| A/S | Exendin Analogs and | WO 2001035988 |
| | GLP-1 Receptor-Binding | Date of Publication: |
| | Non-Peptides | May 25, 2001 |
| National | N-Terminal | Endocrinology (2001), |
| Institutes of | 6-Aminohexanoic Acid | 142(10), 4462-4468. |
| Health | Glucagon-Like Peptide-1 | |
| | Analogue | |
| University of | Glucagon-Like Peptide-1 | Can. Biochemistry |
| Toronto | Analogues | (2001), 40(9), |
| | | 2860-2869. |
| Betagene, Inc. | Heterologous Polypeptides | U.S. Pat. No. 6194176 |
| | | Date of Issue: |
| | | Feb. 27, 2001 |
| Zealand | Peptide Conjugates | PCT Int. Appl. |
| Pharmaceuticals A/S | Containing Variants of | WO 2001004156 |
| | Exendin-4 and GLP-1 | Date of Publication: |
| | | Jan. 18, 2001 |
| Amylin | Exendin and Exendins | PCT Int. Appl. |
| Pharmaceuticals | Agonists | WO 2000073331 |
| | | Date of Publication: |
| | | Dec. 7, 2001 |
| Amylin | Modified Exendins and | PCT Int. Appl. |
| Pharmaceuticals | Exendin Agonists. | WO 2000066629 |
| | | Date of Publication: |
| | | Nov. 9, 2000 |
| Neurogen Corp. | Aryl and Heteroaryl | PCT Int. Appl. |
| | Fused Aminoalkyl- | WO 2000059887 |
| | Imidazoles | Date of Publication: |
| | | Oct. 12, 2000 |
| Amylin | Exendin Agonist | PCT Int. Appl. |
| Pharmaceuticals | Formulations | WO 0041546 |
| | | Date of Publication: |
| | | Jul. 20, 2000 |
| University of | Somatostatin Receptor | American Journal of |
| Toronto | Subtype-5 | Physiology (2000), |
| | | 279(5, Pt. 1), |
| | | G983-G989. |
| Novo Nordisk | GLP-1 Derivatives | PCT Int. Appl. |
| A/S | | WO 9943708 |
| | | Date of Publication: |
| | | Sep. 2, 1999 |
| Novo Nordisk | GLP-1 analogs | PCT Int. Appl. |
| A/S | | WO 9943706 |
| | | Date of Publication: |
| | | Sep. 2, 1999 |
| Novo Nordisk | N-Terminally Truncated | PCT Int. Appl. |
| A/s, | GLP-1 Lipophilic | WO 9943705 |
| | Derivatives | Date of Publication: |
| | | Sep. 2, 1999 |
| Novo Nordisk | GLP-1 Derivatives with | PCT Int. Appl. |
| A/S | Helix-Content Exceeding | WO 9943341 |
| | 25% | Date of Publication: |
| | | Sep. 2, 1999 |
| Amylin | Exendin, Glucagon-like | PCT Int. Appl. |
| Pharmaceuticals | Peptide-1[7-36]amide, | WO 9940788 |
| | or Their Agonists | Date of Publication: |
| | | Aug. 19, 1999 |
| Christian- | Glucagon-like Peptide I | European Journal of |
| Albrechts- | Analogues | Clinical Investigation |
| University | | (1999), 29(7), |
| of Kiel | | 610-614. |
| Pharmacia and | Glucagon-like Peptide-1 | Metabolism, Clinical |
| Upjohn | Receptor Antagonist | and Experimental (1999), |
| | Exendin(9-39) | 48(6), 716-724. |
| Amylin | Exendin Peptides | PCT Int. Appl. |
| Pharmaceuticals | | WO 9830231 |
| | | Date of Publication: |
| | | Jul. 16, 1998 |
| Novo Nordisk | Lipophilic Human | PCT Int. Appl. |
| A/S | Glucagon-like Peptide-1 | WO 9808871 |
| | Derivatives | Date of Publication: |
| | | Mar. 5, 1998 |
| Amylin | Exendin Peptide Analogs | PCT Int. Appl. |
| Pharmaceuticals | | WO 9805351 |
| | | Date of Publication: |
| | | Feb. 12, 1998 |
| Administrators | Linear Somatostatin | U.S. Pat. No. 5,633,263 |
| of the Tulane | Analogs | Date of Issue: |
| Educational | | May 27, 1997 |
| Fund, USA | | |
| Eli Lilly | Glucagon-like | U.S. Pat. No. 5,705,483 |
| | Insulinotropic Peptides | Date of Issue: |
| | | Jan. 6, 1998 |
| Biomeasure, | Cyclic Peptide Analogs | PCT Int. Appl. |
| Incorporated | of Somatostatin. | WO 9711962 |
| | | Date of Publication: |
| | | Apr. 3, 1997 |
| National | Antagonists of | Journal of Biological |
| Institutes of | Glucagon-like Peptide-1 | Chemistry (1997), 272(34), |
| Health | Receptor. | 21201-21206 |
| University of | GLP-1-like Peptides | Proceedings of the |
| Toronto | | National Academy of |
| | | Sciences of the |
| | | United States of |
| | | America (1997), 94(15), |
| | | 7915-7920. |
| University of | Neuropeptide Y | Biomedical Research |
| Shizuoka, | | (1997), 18(2), |
| Shizuoka, Japan. | | 129-137. |
| Kyoto | Human PHI-27 | Chemical & |
| Pharmaceutical | | Pharmaceutical |
| Univ., Kyoto, | | Bulletin (1997), 45(1), |
| Japan. | | 18-26. |
| Eli Lilly | C-Terminal Fragments | Eur. Pat. Appl. |
| | of Glucagon-like | EP 699686 |
| | Insulinotropic Peptide | Date of Publication: |
| | | Mar. 6, 1996 |
| University of | GLP-1 and Related | Can. Endocrine (1995), |
| Toronto | peptides | 3(7), 499-503. |
| Amylin | Amylin Agonists | PCT Int. Appl. |
| Pharmaceuticals | | WO 9310146 |
| | | Date of Publication: |
| | | May 27, 1993 |
| Cent. | Preproglucagon Fragments | Colloque INSERM (1989), |
| Pharma-col.- | | 174(Forum Pept., 2nd, |
| Endocrinol., CNRS, | | 1988), 519-22. |
| Montpellier, Fr | | |
| University of | Iodinated Derivatives | Peptides (New York, |
| Calgary | of Vasoactive Intestinal | NY, United States) |
| | Peptide (VIP), PHI and PHM | (1987), 8(4), 663-76. |
| Univ. Kansas, | Neuropeptide Y Homolog | Biochemical and |
| Kansas City, KS | | Biophysical Research |
| | | Communications (1986), |
| | | 141(3), 1084-1091. |
| Otsuka | Human Peptide Hormones | Japanese Patent |
| Pharmaceutical | | JP 60041698 |
| Co., Ltd., Japan). | | Date of Issue: |
| | | Mar. 5, 1985 |
| ConjuChem | CJC-1131 | |
| Human Genome | Albugon (albumin-based | |
| Sciences | fusion of hGLP-1) | |

Another embodiment includes Acrp30 Substances Used to Treat Diabetes Related Conditions of Table 10.

TABLE 10

| | | Reference to Source |
|---|---|---|
| | | of Acrp30 Compounds |
| Company | Chemical Type | Having Activity |
| Lexigen | Chimeric Proteins | PCT Int. Appl. |
| Pharmaceuticals | | WO 2002072605 |
| | | Date of Issue: |
| | | Sep. 19, 2002 |
| Genset | OBG3 Protein | U.S. Patent. Appl. |
| | Globular Head | Publication |
| | | U.S. Pat. No. 2002091080 |
| | | Date of Publication: |
| | | Jul. 11, 2002 |
| Eli Lilly | Human C1q-Related | PCT Int. Appl. WO |
| | Factor (CRF)-like | 2002012475 |
| | Cerebellin Homolog | Date of Publication: |
| | Protein LP231 | Feb. 14, 2002 |
| Eli Lilly | Cerebellin-like | PCT Int. Appl. WO |
| | Protein LP232 | 2002000709 |
| | | Date of Publication: |
| | | Jan. 3, 2002 |
| Genset | OBG3 Protein | PCT Int. Appl. |
| | Globular Head | WO 2001092330 |
| | | Date of Publication: |
| | | Dec. 6, 2001 |
| | Protein Homolog | PCT Int. Appl. |
| | ACRP30R2 | WO 2001053312 |
| | | Date of Publication: |
| | | Jul. 26, 2001 |
| Genset | OBG3 and gOBG3 | PCT Int. Appl. |
| | Polypeptide | WO 2001051645 |
| | Fragments | Date of Publication: |
| | | Jul. 19, 2001 |
| Osaka | CORS26 Protein | J. Biol. Chem. (2001), |
| University | | 276(5), 3628-3634. |
| Genset | gAcrp30 | Proceedings of National |
| | | Academy of Sciences of |
| | | United States (2001), |
| | | 98(4), 2005-2010. |
| Nanfang Research | C1q Subunit A | Chinese Patent |
| Center, National | Isoform (hC1QA-iso) | CN 1281041 |
| Human Gene Group, | | Date of Issue: |
| PRC | | Jan. 24, 2001 |
| Zymogenetics | Protein Homolog | PCT Int. Appl. |
| | ZACRP7 | WO 2000073448 |
| | | Date of Publication: |
| | | Dec. 7, 2000 |
| SmithKline | Protein Homolog | PCT Int. Appl. |
| Beecham Corp. | ACRP30R1M | WO 2000064943 |
| | | Date of Publication: |
| | | Nov. 2, 2000 |
| Zymogenetics | Protein Homolog | PCT Int. Appl. |
| | ZACRP2 | WO 2000063376 |
| | | Date of Publication: |
| | | Oct. 26, 2000 |
| SmithKline | Protein Homolog | PCT Int. Appl. |
| Beecham Corp. | ACRP30R2 | WO 9964629 |
| | | Date of Publication: |
| | | Dec. 16, 1999 |
| SmithKline | Protein Homolog | PCT Int. Appl. |
| Beecham Corp. | ACRP30R1 | WO 9959619 |
| | | Date of Publication: |
| | | Nov. 25, 1999 |
| SmithKline | Protein Homolog | PCT Int. Appl. |
| Beecham Corp. | ACRP30R1L | WO 9959618 |
| | | Date of Publication: |
| | | Nov. 25, 1999 |
| SmithKline | Human Cerebellin-2 | PCT Int. Appl. |
| Beecham Corp. | Related Proteins | WO 9942576 |
| | | Date of Publication: |
| | | Aug. 26, 1999 |
| Zymogenetics | Protein Homolog | PCT Int. Appl. |
| | ZSIG39 | WO 9910492 |
| | | Date of Publication: |
| | | Mar. 4, 1999 |
| Genset | Lipoprotein- | PCT Int. Appl. |
| | regulating | WO 9907736 |
| | Proteins | Date of Publication: |
| | | Feb. 18, 1999 |
| | Human Homolog | Biochem. Biophys. |
| | Apm-1 | Res. Commun., (1996), |
| | | 221, 286-289. |
| | AdipoQ Peptide | Journal of Biological |
| | Homologs | Chemistry (1996), 271, |
| | | 10697-10703. |
| | GBP28 Peptide | Journal of Biochemistry |
| | Homolog | (Tokyo) (1996), 120, |
| | | 803-812. |
| | ACRP30 Protein | Journal of Biological |
| | Homologs | Chemistry (1995), 270, |
| | | 26746-26749. |

In one embodiment, the aldosterone receptor antagonist is eplerenone and the antidiabetic agent is Metformin (in any form including slow release, etc.); a sulfonylurea; a PPAR gamma agonist with or without additional PPARalpha agonist activity; an injectable insulin; or a Meglitinide analog and other non-sulfonylurea, rapidly acting insulin secretagogues (including repaglinide/Prandin; nateglinide/Starlix; mitiglinide). It is noted that the eplerenone would not be physically combined with injectables, but instead administered separately.

In another embodiment, the aldosterone receptor antagonist is eplerenone and the antidiabetic agent is an agonist of GLP-1 receptor (GLP-1s and related analogs such as Exendin4); a DPP-IV inhibitor; a PPARalpha/gamma dual agonist; an inhaled insulin; an insulin; a PTP-1B inhibitor; or a fructose-1,6-bisphosphatase inhibitors (e.g., Metabasis' CS-917).

In another embodiment, the aldosterone receptor antagonist is eplerenone and the antidiabetic agent is a glucocorticoid antagonist; a glucagon antagonist; an adiponectin/APM1/acrp30 or related analog or fragment thereof; a 11-beta-hydroxysteroid dehydrogenase-1 inhibitor; or a insulin receptor activator (such as Merck's L-783281)

The combination therapy of the invention would be useful in treating a variety of complications of diabetic and prediabetic states including, but not limited to, circulatory disorders, including cardiovascular disorders, such as hypertension, congestive heart failure, myocardial fibrosis and cardiac hypertrophy. The combination therapy would also be useful with adjunctive therapies. For example, the combination therapy may be used in combination with other drugs, such as a diuretic, to aid in treatment of hypertension. The combination therapy would also be useful with adjunctive therapies comprising three or more compounds selected from one or more anti-diabetic agents in combination with one or more aldosterone receptor antagonists.

In addition to the aldosterone receptor antagonist and antidiabetic agent, a third compound may be added to the combination therapy selected from the group consisting of renin inhibitors, , angiotensin II antagonists, angiotensin converting enzyme inhibitors, alpha-adrenergic receptor blockers, beta-adrenergic receptor blockers, calcium channel blockers, endothelin receptor antagonists, endothelin converting enzyme inhibitors, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, IIb/IIIa antagonists such as xemilofiban, and orbofiban.

Suitable angiotensin converting enzyme inhibitors are benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

Indications

Combination therapy will be used to treat or prevent complications of diabetic and prediabetic states. These complications include, but are not limited to, coronary artery disease, hypertension, cardiovascular disease, renal dysfunction, cerebrovascular disease, vascular disease, retinopathy, neuropathy (such as peripheral neuropathy), hyperglycemia, hyperinsulinemia and insulin resistance, edema, endothelial dysfunction, baroreceptor dysfunction, and the like. Cardiovascular disease includes, but is not limited to, coronary artery disease, heart failure (such as congestive heart failure), arrhythmia, diastolic dysfunction (such as left ventricular diastolic dysfunction, diastolic heart failure, and impaired diastolic filling), systolic dysfunction, ischemia, sudden cardiac death, myocardial and vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, fibrinoid necrosis of coronary arteries, and the like. Renal dysfunction includes, but is not limited to, glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary (endothelial and mesangial) and/or extracapillary cells (crescents), expansion of reticulated mesangial matrix with or without significant hypercellularity, malignant nephrosclerosis (such as ischemic retraction, thrombonecrosis of capillary tufts, arteriolar fibrinoid necrosis, and thrombotic microangiopathic lesions affecting glomeruli and microvessels), and the like. Cerebrovascular disease includes, but is not limited to stroke. Vascular disease includes, but is not limited to, thrombotic vascular disease (such as mural fibrinoid necrosis, extravasation and fragmentation of red blood cells, and luminal and/or mural thrombosis), proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction, and the like. Edema includes, but is not limited to, peripheral tissue edema, hepatic congestion, splenic congestion, liver ascites, respiratory or lung congestion, and the like. Hyperglycemia, hyperinsulinemia and insulin resistance include, but are not limited to, insulin resistance, Type I diabetes mellitus, Type II diabetes mellitus, glucose intolerance, pre-diabetic state, metabolic syndrome, and the like.

The combination therapy is particularly useful for complications selected from the group consisting of coronary artery disease, hypertension, cardiovascular disease, renal dysfunction, edema, cerebrovascular disease, and hyperglycemia, hyperinsulinemia and insulin resistance; more preferably, the pathogenic effects are selected from the group consisting of coronary artery disease, hypertension, cardiovascular disease, stroke, and Type II diabetes mellitus; and still more preferably, the pathogenic effects are selected from the group consisting of coronary artery disease, hypertension, heart failure (particularly heart failure post myocardial infarction), left ventricular hypertrophy, and stroke.

In one embodiment of the present invention, therefore, the method comprises administering a therapeutically-effective amount of one or more epoxy-steroidal compounds that are aldosterone receptor antagonists to treat or prevent one or more aldosterone-mediated pathogenic effects in a human subject suffering from or susceptible to the pathogenic effect or effects, wherein the subject has a sub-normal endogenous aldosterone level. The pathogenic effect or effects preferably are selected from the group consisting of hypertension, cardiovascular disease, cerebrovascular disease, and Type II diabetes mellitus; and more preferably, the pathogenic effects are selected from the group consisting of hypertension, heart failure (particularly heart failure post myocardial infarction), left ventricular hypertrophy, and stroke. The epoxy-steroidal compound preferably is eplerenone.

Patients or Subjects of Treatment

The patients or subjects of the treatment or prophylaxis of the invention include diabetics (Type I and Type II); subjects with impaired glucose tolerance, subjects having impaired fasting glucose, subjects with metabolic syndrome (syndrome X), subjects having a family history of diabetes, and diabetics who cannot adequately control glucose levels with insulin.

Metabolic syndrome symptoms can include obesity/abdominal obesity, frank diabetes, hypertension, dyslipidemia (hypertriglyceridemia, low HDL-cholesterol, and/or smaller and more atherogenic forms of LDL-cholesterol, etc.), insulin resistance, microalbuminuria, and a hypercoagulable state. The patients or subjects may also include those having salt sensitivity and/or an elevated dietary sodium intake. See for example, Earl S. Ford, et al., JAMA, Jan. 16, 2002, Vol. 287, No. 3, pp 356-359. See also L. Groop et al., “The Dysmetabolic Syndrome” Journal of Internal Medicine 2001; 250: 105-120.

Definitions

The term “hydrido” denotes a single hydrogen atom (H). This hydrido group may be attached, for example, to an oxygen atom to form a hydroxyl group; or, as another example, one hydrido group may be attached to a carbon atom to form a 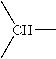
group; or, as another example, two hydrido atoms may be attached to a carbon atom to form a —CH₂— group. Where the term “alkyl” is used, either alone or within other terms such as “haloalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The term “cycloalkyl” embraces cyclic radicals having three to about ten ring carbon atoms, preferably three to about six carbon atoms, such as cyclopropyl, cyclobutyl, cyclopentyl and cyclohexyl. The term “haloalkyl” embraces radicals wherein any one or more of the alkyl carbon atoms is substituted with one or more halo groups, preferably selected from bromo, chloro and fluoro. Specifically embraced by the term “haloalkyl” are monohaloalkyl, dihaloalkyl and polyhaloalkyl groups. A monohaloalkyl group, for example, may have either a bromo, a chloro, or a fluoro atom within the group. Dihaloalkyl and polyhaloalkyl groups may be substituted with two or more of the same halo groups, or may have a combination of different halo groups. A dihaloalkyl group, for example, may have two fluoro atoms, such as difluoromethyl and difluorobutyl groups, or two chloro atoms, such as a dichloromethyl group, or one fluoro atom and one chloro atom, such as a fluoro-chloromethyl group. Examples of a polyhaloalkyl are trifluoromethyl, 1,1-difluoroethyl, 2,2,2-trifluoroethyl, perfluoroethyl and 2,2,3,3-tetrafluoropropyl groups. The term “difluoroalkyl” embraces alkyl groups having two fluoro atoms substituted on any one or two of the alkyl group carbon atoms. The terms “alkylol” and “hydroxyalkyl” embrace linear or branched alkyl groups having one to about ten carbon atoms any one of which may be substituted with one or more hydroxyl groups. The term “alkenyl” embraces linear or branched radicals having two to about twenty carbon atoms, preferably three to about ten carbon atoms, and containing at least one carbon-carbon double bond, which carbon-carbon double bond may have either cis or trans geometry within the alkenyl moiety. The term “alkynyl” embraces linear or branched radicals having two to about twenty carbon atoms, preferably two to about ten carbon atoms, and containing at least one carbon-carbon triple bond. The term “cycloalkenyl” embraces cyclic radicals having three to about ten ring carbon atoms including one or more double bonds involving adjacent ring carbons. The terms “alkoxy” and “alkoxyalkyl” embrace linear or branched oxy-containing radicals each having alkyl portions of one to about ten carbon atoms, such as methoxy group. The term “alkoxyalkyl” also embraces alkyl radicals having two or more alkoxy groups attached to the alkyl radical, that is, to form monoalkoxyalkyl and dialkoxyalkyl groups. The “alkoxy” or “alkoxyalkyl” radicals may be further substituted with one or more halo atoms, such as fluoro, chloro or bromo, to provide haloalkoxy or haloalkoxyalkyl groups. The term “alkylthio” embraces radicals containing a linear or branched alkyl group, of one to about ten carbon atoms attached to a divalent sulfur atom, such as a methylthio group. Preferred aryl groups are those consisting of one, two, or three benzene rings. The term “aryl” embraces aromatic radicals such as phenyl, naphthyl and biphenyl. The term “aralkyl” embraces aryl-substituted alkyl radicals such as benzyl, diphenylnethyl, triphenylmethyl, phenyl-ethyl, phenylbutyl and diphenylethyl. The terms “benzyl” and “phenylmethyl” are interchangeable. The terms “phenalkyl” and “phenylalkyl” are interchangeable. An example of “phenalkyl” is “phenethyl” which is interchangeable with “phenylethyl”. The terms “alkylaryl”, “alkoxyaryl” and “haloaryl” denote, respectively, the substitution of one or more “alkyl”, “alkoxy” and “halo” groups, respectively, substituted on an “aryl” nucleus, such as a phenyl moiety. The terms “aryloxy” and “arylthio” denote radicals respectively, provided by aryl groups having an oxygen or sulfur atom through which the radical is attached to a nucleus, examples of which are phenoxy and phenylthio. The terms “sulfinyl” and “sulfonyl”, whether used alone or linked to other terms, denotes, respectively, divalent radicals SO and SO₂. The term “aralkoxy”, alone or within another term, embraces an aryl group attached to an alkoxy group to form, for example, benzyloxy. The term “acyl” whether used alone, or within a term such as acyloxy, denotes a radical provided by the residue after removal of hydroxyl from an organic acid, examples of such radical being acetyl and benzoyl. “Lower alkanoyl” is an example of a more preferred sub-class of acyl. The term “amido” denotes a radical consisting of nitrogen atom attached to a carbonyl group, which radical may be further substituted in the manner described herein. The term “monoalkylaminocarbonyl” is interchangeable with “N-alkylamido”. The term “dialkylaminocarbonyl” is interchangeable with “N,N-dialkylamido”. The term “alkenylalkyl” denotes a radical having a double-bond unsaturation site between two carbons, and which radical may consist of only two carbons or may be further substituted with alkyl groups which may optionally contain additional double-bond unsaturation. The term “heteroaryl”, where not otherwise defined before, embraces aromatic ring systems containing one or two heteroatoms selected from oxygen, nitrogen and sulfur in a ring system having five or six ring members, examples of which are thienyl, furanyl, pyridinyl, thiazolyl, pyrimidyl and isoxazolyl. Such heteroaryl may be attached as a substituent through a carbon atom of the heteroaryl ring system, or may be attached through a carbon atom of a moiety substituted on a heteroaryl ring-member carbon atom, for example, through the methylene substituent of imidazolemethyl moiety. Also, such heteroaryl may be attached through a ring nitrogen atom as long as aromaticity of the heteroaryl moiety is preserved after attachment. For any of the foregoing defined radicals, preferred radicals are those containing from one to about ten carbon atoms.

Specific examples of alkyl groups are methyl, ethyl, n-propyl, isopropyl, n-butyl, sec-butyl, isobutyl, tert-butyl, n-pentyl, isopentyl, methylbutyl, dimethylbutyl and neopentyl. Typical alkenyl and alkynyl groups may have one unsaturated bond, such as an allyl group, or may have a plurality of unsaturated bonds, with such plurality of bonds either adjacent, such as allene-type structures, or in conjugation, or separated by several saturated carbons.

Racemates, Stereoisomers, and Salts Thereof

As noted above, the aldosterone receptor antagonists and anti-diabetic agents useful in the present combination therapy also may include the racemates and stereoisomers, such as diastereomers and enantiomers, of such agents. Such stereoisomers can be prepared and separated using conventional techniques, either by reacting enantiomeric starting materials, or by separating isomers of compounds of the present invention. Isomers may include geometric isomers, for example cis isomers or trans isomers across a double bond. All such isomers are contemplated among the compounds of the present invention. Such isomers may be used in either pure form or in admixture with those agents described above. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention.

The compounds useful in the present invention as discussed below include their salts, solvates and prodrugs. The compounds useful in the present invention also include tautomers. The term “pharmaceutically-acceptable salts” embraces salts commonly used to form alkali metal salts and to form addition salts of free acids or free bases.

The nature of the salt is not critical, provided that it is pharmaceutically-acceptable.

Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid.

Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, flumaric, pyruvic, aspartic, glutamic, benzoic, anthranic,p-hydroxybenzoic, salicyclic, phenylacetic, mandelic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, b-hydroxybutyric, malonic, galactaric and galacturonic acid. Suitable pharmaceutically-acceptable base addition salts include metallic salts made from aluminium, calcium, lithium, magnesium, potassium, sodium and zinc or organic salts made from N,N′-dibenzylethylenediamine, chloroprocaine, choline, diethanolamine, ethylenediamine, meglumine (N-methylgluca-mine) and procaine. All of these salts may be prepared by conventional means from the corresponding compound by reacting, for example, the appropriate acid or base with such compound.

Mechanism of Action

Multiple large epidemiological studies have suggested that insulin resistance, even in the absence of frank diabetes, is a predictor of coronary artery disease (J E Reusch, Am. J. Cardiol. 90(suppl): 19G-26G, 2002). In general these studies have shown a relationship between plasma insulin levels (a surrogate marker of insulin resistance) and cardiovascular disease. For example, the Helsinki Policemen Study (Balkau B. Shipley M. Jarrett R J. Pyorala K. Pyorala M. Forhan A. Eschwege E. Diabetes Care. 21(3):360-7, March 1998 demonstrated that the incidence of cardiovascular mortality, nonfatal MI, and other cardiovascular events was associated with increasing plasma insulin levels.

The Metabolic Syndrome is characterized by the presence of multiple cardiovascular risk factors and metabolic abnormalities such as obesity, hyperinsulinemia, hypertriglyceridemia, reduced HDL-cholesterol, and hypertension. In comparison to individuals with normal glucose tolerance, prevalence of the Metabolic Syndrome increases in patients with impaired glucose tolerance or impaired fasting glucose, and is even more common in patients with Type 2 diabetes. The presence of the Metabolic Syndrome increases the risk for developing cardiovascular disease and cardiovascular mortality (B Isomaa et al., Diabetes Care 24: 683-689, 2001). The prevalence of CHD, MI, and stroke are all substantially elevated in individuals displaying the Metabolic Syndrome, compared to those without the syndrome. Insulin resistance, hypertension, and microalbuminuria are amongst the important predictors of cardiovascular morbidity and mortality in this syndrome.

The presence of frank diabetes substantially increases the risk of cardiovascular morbidity and mortality (J B Marks and P Raskin, Journal of Diabetes and its Complications 14: 108-115, 2000). Cardiovascular disease is increased in both Type I and Type II diabetics compared to the nondiabetic population, and the extent of cardiovascular disease is related to the severity of hyperglycemia. The primary cause of mortality in the diabetic population is cardiovascular disease.

Hypertension is approximately twice as common in the diabetic population as compared to the nondiabetic population, as is the incidence of isolated systolic hypertension. Importantly, diabetes and hypertension are independent predictors of cardiovascular mortality. Tight control of blood pressure reduces cardiovascular risk to a greater extent in diabetics as compared to nondiabetics. In hypertensive individuals, diabetes further increases the risk of developing heart failure. Diabetes may predispose patients to develop heart failure in the presence of well-known cardiovascular risk factors such as hypertension and coronary artery disease.

Given the independent effects of insulin resistance or diabetes and those of hypertension to accelerate the development of cardiovascular disease, it is anticipated that combining the effects of aldosterone receptor blockade with standard antidiabetic therapy should ameliorate the progression of cardiovascular complications in the insulin-resistant or diabetic state in comparison to the effects of either treatment alone. It is now well-documented via large intervention trials such as the Diabetes Control and Complications Trial and the United Kingdom Prospective Diabetes Study that reduction of hyperglycemia in both Type I and Type II diabetes, via intensive insulin therapy or treatment with oral antidiabetic agents, reduces the complications of diabetes. In particular, improvements in long-term glycemic control have been shown to significantly reduce the onset and progression of diabetic neuropathy and microvascular complications such as nephropathy and retinopathy. The effects of intensive glycemic control on macrovascular complications have been more difficult to document. Combination therapy with aldosterone receptor antagonists, which have documented beneficial effects on the macrovasculature, as well as the microvasculature, will be clinically important in diabetics. It is well accepted that antihypertensive agents reduce the progression of nephropathy and cardiovascular disease in the general population and specifically in diabetics. Preclinical and clinical studies further suggest that aldosterone receptor blockade can ameliorate the development of diabetic complications. For example, in experimentally-induced diabetes, treatment with the aldosterone receptor antagonist spironolactone, in the absence of any antidiabetic therapy, reduces the detrimental deposition of collagen and fibronectin in the heart, kidneys and vasculature and lessens the development of passive diastolic stiffness (P. E. White et al., Endocrine Reviews, Vol. 18, No. 1, pp. 135-156 (1997).

Currently available data suggest that aldosterone receptor blockade will provide significant advantages over existing antihypertensive therapy in the diabetic setting. Angiotensin converting enzyme inhibitors (ACEi) are currently used to retard the progression of nephropathy in nondiabetic and diabetic patients. In a significant number of patients, chronic treatment with ACEi results over time in a diminished ability to block the renin-angiotensin-aldosterone system, such that over time aldosterone levels begin to rise despite continued drug treatment (commonly referred to as “aldosterone escape”). A recent study of diabetics with early nephropathic changes demonstrated that aldosterone escape can occur in a substantial proportion of diabetic patients, and that patients experiencing the escape phenomenon show more severe deterioration in indices of renal function (A. Sato et al., Hypertension 41: 64-68, 2003). Subsequent addition of spironolactone to the treatment regimen (i.e. in the presence of continuing ACEi therapy) of patients experiencing aldosterone escape resulted in a substantial reduction in indices of both left ventricular hypertrophy and nephropathy. These changes were observed in the absence of any further diminution of blood pressure compared to the effects of ACEi alone, demonstrating the potential for aldosterone receptor blockade to exert beneficial macrovascular and microvascular effects independent of antihypertensive action.

In the kidney, mineralocorticoid receptors can be activated by either mineralocorticoids (e.g. aldosterone) or glucocorticoids (e.g. cortisol). Normally, cortisol (which is present in the circulation at much higher concentrations than aldosterone) does not activate the mineralocorticoid receptor due to the presence in the kidney of the enzyme 11-beta-hydroxysteroid dehydrogenase-type 2 (11betaHSD2). 11betaHSD2 metabolizes and inactivates glucocorticoids, preventing them from binding to the mineralocorticoid receptor. In the rare but clinically important condition of Apparent Mineralocorticoid Excess, mutations of 11betaHSD2 that diminish its activity allow cortisol access to the mineralocorticoid receptor, resulting in sodium retention, hypokalemia, and hypertension (P. M. Stewart et al., J. Clin. Invest. 82: 340-349, 1988). In an experimental model of diabetes characterized by increases in blood pressure, renal levels of 11betaHSD2 were reduced. Insulin therapy lowered blood pressure to normal and restored the levels of renal 11betaHSD2 (Y.-J. Liu et al., Hypertension 31: 885-889, 1998), suggesting that the reduction in 11betaHSD2 activity results in abnormal activation of the renal mineralocorticoid receptor by circulating cortisol. Aldosterone receptor blockade in the absence of antidiabetic therapy also normalizes blood pressure and 11betaHSD2 levels in experimental diabetes (Y.-J. Liu et al., Kid. Intl. 57: 2064-2071, 2000). It is reasonable to suggest that the effects of antidiabetic therapy and aldosterone receptor blockade may be synergistic in lowering blood pressure in the diabetic state.

In an in vitro model of cardiac hypertrophy, aldosterone has been shown to stimulate surrogates of hypertrophy in a process mediated via the mineralocorticoid receptor (A. Sato and J. W. Funder, Endocrinology 137: 4145-4153, 1996). In this setting, hyperglycemia by itself does not stimulate hypertrophy, but interacts synergistically with aldosterone to promote hypertrophy. This synergistic effect can be prevented by aldosterone receptor blockade. It is reasonable that the interactions of diabetes and hypertension to promote macrovascular disease can be prevented in a synergistic fashion by combining antidiabetic therapy to lower blood glucose levels with selective aldosterone receptor blockade.

The progression of atherosclerotic disease is believed to be due in part to a proinflammatory state (P M Ridker et al., New Eng. J. Med. 347: 1557-1565, 2002). It is now also recognized that states of obesity, insulin resistance and diabetes are characterized by increased oxidative stress and inflammation. The proinflammatory state in diabetes may contribute to the underlying insulin resistance (M Yuan et al., Science 293: 1673-1677, 2001) as well as to the enhanced rates of atherosclerosis and renal dysfunction. In recent years some of the beneficial cardiovascular effects of the lipid-lowering statin class of drugs (inhibitors of HMG-CoA reductase) and the antidiabetic PPARgamma agonists have been ascribed to their additional anti-inflammatory actions (P Dandona and A Aljada, Am. J. Cardiol. 90(suppl): 27G-33G, 2002). Given that aldosterone antagonism has been shown to have pronounced anti-inflammatory effects in tissues susceptible to diabetic complications such as the peripheral vasculature, kidney and heart, aldosterone antagonism is predicted to be particularly suited to inhibit the progression of diabetic vascular complications.

In recent years it has become evident that adipose tissue synthesizes and secretes a number of proteins that have actions in the vasculature, such as plasminogen activator inhibitor-1 (B E Sobel, Am. J. Med. 113(6A): 12S-22S, 2002), angiotensinogen (S Engali et al., Hypertension 35: 1270-1277, 2000), and adiponectin (T Yamauchi et al., J. Biol. Chem. 278: 2461-2468, 2003). Adipose tissue expression of these proteins is dysregulated in obesity and in the diabetic state. Furthermore, adipose tissue appears to express the key components of the renin-angiotensin system. It has been hypothesized that adipose tissue production of angiotensin may contribute to hypertension often seen in obesity and Type II diabetes (K Gorzelniak et al., J.

Hypertension 20: 965-973, 2002). Given that the RAS system activates aldosterone synthesis, aldosterone receptor antagonists may prove beneficial in neutralizing adverse effects of adipose tissue activation of the RAS system in states of insulin resistance and diabetes.

Advantages of Combination Therapy

The selected aldosterone receptor antagonists and anti-diabetic agent of the present invention act in combination to provide more than an additive benefit. For example, administration of an aldosterone receptor antagonist and anti-diabetic agent combination can result in the near-simultaneous reduction in pathogenic effects of multiple risk factors for diabetic complications such as nephropathy and atherosclerosis. For example, drug combinations may reduce several risk factors for atherosclerosis, such as high aldosterone levels, high blood pressure, endothelial dysfunction, hyperglycemia, insulin resistance, glycated proteins and lipoproteins, low HDL-cholesterol, elevated plasma triglycerides, more atherogenic subfractions of LDL-cholesterol, vascular inflammation, a prothrombotic state, etc. The distinct risk factors affected by each combination will depend on the mechanism of a given anti-diabetic agent. Synergy may also result from combination therapy if some of the deleterious effects of aldosterone are potentiated by the diabetic state, e.g. if levels of the enzyme 11-beta-hydroxysteroid dehydrogenase-type 2 are reduced in the diabetic state, or if effects of aldosterone to stimulate cardiac hypertrophy are potentiated by hyperglycemia. Simultaneous amelioration of 11-beta-hydroxysteroid dehydrogenase-type 2 activity (or reduction in glycemia) and aldosterone receptor blockade may provide synergy.

The methods of this invention also provide for the effective prophylaxis and/or treatment of pathological conditions with reduced side effects compared to conventional methods known in the art. For example, administration of anti-diabetic agents can result in side effects such as, but not limited to, hypoglycemia, hepatic injury, edema, increased adiposity, nausea, and gastrointestinal distress. Reduction of the anti-diabetic agent doses in the present combination therapy below conventional monotherapeutic doses will minimize, or even eliminate, the side-effect profile associated with the present combination therapy relative to the side-effect profiles associated with, for example, monotherapeutic administration of anti-diabetic agents. The side effects associated with anti-diabetic agents typically are dose-dependent and, thus, their incidence increases at higher doses. Accordingly, lower effective doses of anti-diabetic agents will result in fewer side effects than seen with higher doses of anti-diabetic agents in monotherapy or decrease the severity of such side effects.

Other benefits of the present combination therapy include, but are not limited to, the use of a selected group of aldosterone receptor antagonists that provide a relatively quick onset of therapeutic effect and a relatively long duration of action. For example, a single dose of one of the selected aldosterone receptor antagonists may stay associated with the aldosterone receptor in a manner that can provide a sustained blockade of aldosterone receptor activation. Because diabetic complications result from chronic exposure to risk factors such as hypertension and hyperglycemia, more sustained reduction in risk factor profiles is expected to enhance the treatment effect. Another benefit of the present combination therapy includes, but is not limited to, the use of a selected group of aldosterone receptor antagonists, such as the epoxy-steroidal aldosterone receptor antagonists exemplified by eplerenone, which act as highly selective aldosterone receptor antagonists, with reduced side effects that can be caused by aldosterone receptor antagonists that exhibit non-selective binding to non-mineralocorticoid receptors, such as androgen and progesterone receptors. The use of selective aldosterone blockers is expected to reduce the incidence of side effects such as impotence, gynecomastia, and breast pain.

Further benefits of the present combination therapy include, but are not limited to, the use of the methods of this invention to treat individuals who belong to one or more specific racial or ethnic groups that are particularly responsive to the disclosed therapeutic regimens. Thus, for example, individuals of African, native American, or Hispanic ancestry may particularly benefit from the combination therapy of an aldosterone receptor antagonist and an anti-diabetic agent to treat or prevent diabetic vascular complications. The incidence and prevalence of diabetic complications varies amongst different racial and ethnic groups (reference: Diabetes 2001: Vital Statistics, published by the American Diabetes Association, copyright 2001). For example, the incidence of diabetic end stage renal disease is 4-6 times higher in African Americans, Native Americans, and Mexican Americans than non-Hispanic whites. Diabetes-related peripheral vascular disease is more prevalent in Mexican Americans than non-Hispanic whites, and diabetes-related limb amputations are higher in African Americans that whites. The prevalence of diabetic retinopathy is higher in African Americans and Mexican Americans compared to non-Hispanic white Americans with the prevalence of blindness twice as high in African American as whites. Overall, age-adjusted diabetes mortality rates are higher for African Americans, Hispanic Americans, and Native Americans compared to non-Hispanic whites. Because aldosterone receptor blockade is more efficacious in controlling hypertension in some of these same racial/ethnic groups, e.g. in African Americans, it is reasonable to expect that combination therapy will be more efficacious in controlling diabetes-related complications and their associated morbidity and mortality. See Pratt J H, et al. Flack J M et al. Efficacy and tolerability of eplerenone and losartan in hypertensive black and white patients. J Am Coll Cardiol 2003; 41:1148-1155.

Kits

The present invention further comprises kits that are suitable for use in performing the methods of treatment and/or prevention described above. In one embodiment, the kit contains a first dosage form comprising one or more of the aldosterone receptor antagonists identified in Table 1 and a second dosage form comprising one or more of the anti-diabetic agents and agents used in treating the symptoms and conditions associated with diabetes identified in Tables 2-10 in quantities sufficient to carry out the methods of the present invention. Preferably, the first dosage form and the second dosage form together comprise a therapeutically effective amount of the inhibitors for the treatment or prevention of a diabetic condition.

In another embodiment, the kit contains a first dosage form comprising the aldosterone receptor antagonist spironolactone and a second dosage form comprising an anti-diabetic agent and agents used in treating the symptoms and conditions associated with diabetes identified in Tables 2-10 in quantities sufficient to carry out the methods of the present invention.

In another embodiment, the kit contains a first dosage form comprising the aldosterone receptor antagonist eplerenone and a second dosage form comprising an anti-diabetic agent and agents used in treating the symptoms and conditions associated with diabetes identified in Tables 2-10 in quantities sufficient to carry out the methods of the present invention.

Biological Evaluation

In order to determine the probable effectiveness of a combination therapy for diabetes and related conditions and symptoms, it is important to determine the potency of components in several assays. Accordingly, in Assay “A” the activity of an anti-diabetic agent can be determined. In Assay “B,” a method is described for evaluating a combination therapy of the invention, namely, anti-diabetic agent and an epoxy-steroidal aldosterone receptor antagonist. The efficacy of the individual drugs, eplerenone, and anti-diabetic agent, and efficacy of these drugs given together at various doses, are evaluated in rodent models of hypertension and diabetes and related conditions and symptoms.

Therapy Protocols

Preclinical and clinical evaluation of a combination of eplerenone and an antidiabetic agent include, for example, blood pressure measurements, renal function measurements, and glycemic control measurements (plasma glucose, HbA1C, and insulin).

Preclinical Trials

Animal Models: A number of different animal models of obesity, insulin resistance and diabetes are known that also display features of diabetic complications. For example, db/db mice (e.g. M. P. Cohen et al., Exp. Nephrol. 4: 166-171, 1996) and KKAy mice (K Ina et al., Diabetes Research and Clinical Practice 44: 1-8, 1999) are spontaneously obese and diabetic, and develop hypertriglyceridemia, hypercholesterolemia and renal complications reminiscent of diabetic nephropathy.. Fatty Zucker (fa/fa) rats are obese, insulin resistant and hypertensive, and hypertension can be exacerbated by placing animals on a high salt diet (S H Carlson et al., Hypertension 35 (1, Part 2) (Supplement):403, 2000). The Spontaneous Hypertension Heart Failure (SHHF) rat is obese, insulin-resistant, hyperlipidemic, and develops hypertension and heart failure (S. A. McCune et al., Renal and heart function in the SHHF/Mcc-cp rat. In: E Shafrir (editor): Frontiers in diabetes research. Lessons from animal diabetes III. Smith Gordon, London, 1990, pp. 397-401).

Nondiabetic or diabetic animals would be treated with or without therapy for a period of several months, and the effect of therapy on indices of diabetes (plasma glucose and insulin levels, hemoglobin A1c levels) would be measured along with indices of diabetic renal disease, such as albuminuria, renal mesangial expansion, and the increased renal expression of fibronectin and Type IV collagen that occur in diabetes. The following experimental groups could be studied in order to determine whether combination therapy is more efficacious on renal diabetic disease than monotherapy:
Diabetic mice with vehicle treatment
Diabetic mice treated with an antihyperglycemic agent (e.g. PPARgamma agonists)
Diabetic mice treated with eplerenone
Diabetic mice treated with the combination of the antihyperglycemic agent and eplerenone

Clinical Trials

In addition, clinical trials can be used to evaluate aldosterone receptor antagonist therapy in humans. Numerous examples of such therapeutic tests have been published, including those of the RALES 003 study described in American Journal of Cardiology 78, 902-907 (1996) and the RALES 004 study described in New England Journal of Medicine 341, 709-717 (1999).

Clinical trials used to evaluate anti-diabetic agents in humans have also been published. A protocol for blood pressure measurements can be found in Reddi et al., Hypertension 233-238 (August 2000). A protocol for renal function measurement can be found in Epstein et al. “Eplerenone reduces proteinuria in type II diabetes mellitus: Implications for aldosterone involvement in the pathogenesis of renal dysfunction (021)” J Am Coll Cardiol 2002;39(5):Suppl A. In Dr. Edmund J. Lewis at al., N Engl J. Med, Vol 345, No. 12, Sep. 20, 2001, a similar study was performed but with longer treatment and instead of a surrogate endpoint for reduced progression of renal disease (decrease in microalbuminuria), hard endpoints (the doubling of baseline creatine and development of end stage renal disease) were measured.

Other resources include M. Epstein, G. Williams, V. Buckalew, J. Altamirano, B. Roniker, S. Krause and J. Kleiman, “The Selective Aldosterone Blocker Eplerenone Reduces Proteinuria in Hypertensive Patients with Type 2 Diabetes Mellitus,” (preprint submitted in Information Disclosure Statement filed herewith) and Lewis et al., “The Effect of Angiotensin-Converting-Enzyme Inhibition on Diabetic Nephropathy” New England Journal of Medicine Vol. 329:1456-1462 Nov. 11, 1993 No. 20.

After a baseline antidiabetic therapy, patients would be treated with or without eplerenone. The results would be evaluated to determine whether eplerenone addition to antidiabetic therapy reduced complications more than antidiabetic therapy alone. Measures of efficacy would include proteinuria (urinary albumin-to-creatinine ratio), blood pressure, plasma glucose and insulin, and HbA1c.

Administration

Administration of the anti-diabetic agent and the aldosterone receptor antagonist may take place sequentially in separate formulations, or may be accomplished by simultaneous administration in a single formulation or separate formulations. Administration may be accomplished by oral route, or by intravenous, intramuscular or subcutaneous injections. The formulation may be in the form of a bolus, or in the form of aqueous or non-aqueous isotonic sterile injection solutions or suspensions. These solutions and suspensions may be prepared from sterile powders or granules having one or more pharmaceutically-acceptable carriers or diluents, or a binder such as gelatin or hydroxypropyl-methyl cellulose, together with one or more of a lubricant, preservative, surface-active or dispersing agent.

Typically, the aldosterone receptor antagonist is administered in a daily dose ranging from about 0.1 to 2000 mg, and the anti-diabetic agent is administered in a daily dose ranging from about 0.1 to 1000 mg. If included, the angiotensin converting enzyme inhibitor is administered in a daily dose ranging from about 0.1 to 1000 mg.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. These may with advantage contain an amount of each active ingredient from about 1 to 250 mg, preferably from about 25 to 150 mg. A suitable daily dose for a mammal may vary widely depending on the condition of the patient and other factors. However, a dose of from about 0.01 to 30 mg/kg body weight, particularly from about 1 to 15 mg/kg body weight, may be appropriate.

The active ingredients may also be administered by injection as a composition wherein, for example, saline, dextrose or water may be used as a suitable carrier. A suitable daily dose of each active component is from about 0.01 to 15 mg/kg body weight injected per day in multiple doses depending on the disease being treated. A preferred daily dose would be from about 1 to 10 mg/kg body weight. Compounds indicated for prophylactic therapy will preferably be administered in a daily dose generally in a range from about 0.1 mg to about 15 mg per kilogram of body weight per day. A more preferred dosage will be a range from about 1 mg to about 15 mg per kilogram of body weight. Most preferred is a dosage in a range from about 1 to about 10 mg per kilogram of body weight per day. A suitable dose can be administered, in multiple sub-doses per day. These sub-doses may be administered in unit dosage forms. Typically, a dose or sub-dose may contain from about 1 mg to about 100 mg of active compound per unit dosage form. A more preferred dosage will contain from about 2 mg to about 50 mg of active compound per unit dosage form. Most preferred is a dosage form containing from about 3 mg to about 25 mg of active compound per unit dose.

In combination therapy, the anti-diabetic agent may be present in a range of doses, depending on the particular agent used, inherent potency, bioavailabilty and metabolic stability of the composition and whether it has been formulated for immediate release or extended release. Non-limiting examples of dose form ranges for specific anti-diabetic agents are listed below:

| COMPOUND | DOSAGE FORM | STRENGTH RANGE |
|---|---|---|
| Actos | Tablets, oral | 15 mg, 30 mg, 45 mg |
| Amaryl | Tablets, oral | 1 mg, 2 mg, 4 mg |
| Avandia | Tablets, oral | 2 mg, 4 mg, 8 mg |
| Diabeta | Tablets, oral | 1.25 mg, 2.5 mg, 5 mg |
| Glucophage | Tablets, oral | 500 mg, 850 mg, 1000 mg |
| Glucophage XR | Extended-release | 500 mg |
| | tablets, oral | |
| Glucotrol | Scored tablets, | 2.5 mg, 5 mg, 10 mg |
| | oral | |
| Glucotrol XL | Tablets, oral | 2.5 mg, 5 mg, 10 mg |
| Glucovance | Tablets: Glyburide- | 1.25 mg-250 mg, |
| | metformin, oral | 2.5 mg-550 mg, |
| | | 5 mg-500 mg |
| Glynase PresTab | Tablets, oral | 1.5 mg, 3 mg, 6 mg |
| Glyset | Tablets, oral | 25 mg, 50 mg, 100 mg |
| Micronase | Tablets, oral | 1.25 mg, 2.5 mg, 5 mg |
| Prandin | Tablets, oral | 0.5 mg, 1 mg, 2 mg |
| Precose | Tablets, oral | 25 mg, 50 mg, 100 mg |
| Starlix | Tablets, oral | 60 mg, 120 mg |
| Humalog | Injection | 100 units/mL, in 10 mL |
| | | vials, 1.5 mL, 3 mL |
| | | cartridges, 3 mL |
| | | disposable insulin |
| | | delivery device |
| Humalog 50/50 | Injection | 100 units/mL (50% |
| | | insulin lispro protamine, |
| | | 50% insulin lispro), in |
| | | 10 mL vials, 3 mL |
| | | cartridges, 3 mL |
| | | disposable pens |
| Humalog 75/25 | Injection | 100 units/mL (75% |
| | | insulin lispro protamine, |
| | | 25% insulin lispro), in |
| | | 10 mL vials, 3 mL |
| | | cartridges, 3 mL |
| | | disposable pens |
| Humulin 50/50 | Injection | 100 units/mL; 10 mL vials |
| Humulin 75/25 | Injection | 100 units/mL; 10 mL vials |
| Humulin L | Injection | 100 units/mL; 10 mL vials |
| Humulin N | Injection | 100 units/mL; 10 mL vials |
| Humulin R | Injection | 100 units/mL; 10 mL vials |
| Humulin R U-500 | Injection | 500 units/mL; 20 mL vials |
| HumulinU | Injection | 100 units/mL; 10 mL vials |
| Iletin II Lente | Injection | 100 units/mL; 10 mL vials |
| Iletin II NPH | Injection | 100 units/mL; 10 mL vials |
| Iletin II Regular | Injection | 100 units/mL; 10 mL vials, |
| | | 500 units/mL; 10 mL vials |
| Lantus | Solution, | 100 units/mL, in 5 mL, |
| | injection | 10 mL vials, 3 mL |
| | | cartridges for Optipen |
| | | One Insulin Delivery |
| | | Device |
| Novolin L | Injection | 100 units/mL |
| Novolin N | Injection | 100 units/mL |
| Novolin R | Injection | 100 units/mL |
| Novolog | Injection | 100 units/mL |
| Velosulin BR | Injection | 100 units/mL, in 10 mL |
| | | vials and for infusion |
| | | pump |

In combination therapy, the aldosterone receptor antagonist may be present in an amount in a range from about 5 mg to about 400 mg, and the anti-diabetic agent may be present in an amount in a range from about 1 mg to about 10,000 mg, which represents aldosterone receptor antagonist-to-anti-diabetic agent ratios ranging from about 400:1 to about 1:2,000.

In a preferred combination therapy, the aldosterone receptor antagonist may be present in an amount in a range from about 10 mg to about 200 mg, and the anti-diabetic agent may be present in an amount in a range from about 5 mg to about 5,000 mg, which represents aldosterone receptor antagonist-to-anti-diabetic agent ratios ranging from about 40:1 to about 1:500.

In a more preferred combination therapy, the aldosterone receptor antagonist may be present in an amount in a range from about 20 mg to about 100 mg, and the anti-diabetic agent may be present in an amount in a range from about 4,000 mg to about 80 mg, which represents aldosterone receptor antagonist-to-anti-diabetic agent ratios ranging from about 10:1 to about 1:200

Other exemplary anti-diabetic agent doses include, but are not limited to, 9,500 mg, 8,000 mg, 7,000, 6,000 mg, 5,000 mg, 4,000 mg, 3,000 mg, 2,000 mg, 1,500 mg, 1,000 mg, 500 mg, 400 mg, 300 mg, 200 mg, 100 mg, respectively, in combination with an aldosterone antagonist provided in any one of the above-noted aldosterone antagonist dosage ranges specified in previous paragraphs.

The dosage regimen for treating a disease condition with the combination therapy of this invention is selected in accordance with a variety of factors, including the type, age, weight, sex and medical condition of the patient, the severity of the disease, the route of administration, and the particular compound employed, and thus may vary widely.

For therapeutic purposes, the active components of this combination therapy invention are ordinarily combined with one or more adjuvants appropriate to the indicated route of administration. If administered per os, the components may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The components may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The present invention further comprises kits that are suitable for use in performing the methods of treatment and/or prophylaxis described above. In one embodiment, the kit contains a first dosage form comprising one or more of the epoxy-steroidal aldosterone receptor antagonists previously identified and a second dosage form comprising a anti-diabetic agent identified in Table 2 in quantities sufficient to carry out the methods of the present invention. Preferably, the first dosage form and the second dosage form together comprise a therapeutically effective amount of the inhibitors.

Crystalline Forms of Active Compounds

Crystalline forms that are easily handled, reproducible in form, easily prepared, stable, and which are non-hygroscopic have been identified for the aldosterone antagonist eplerenone. These include Form H, Form L, various crystalline solvates and amorphous eplerenone. These forms, methods to make these forms, and use of these forms in preparing compositions and medicaments, are disclosed in Barton et al., WO 01/41535 and Barton et al., WO 01/42272; incorporated herein in their entirety.

In one embodiment of the present invention, the aldosterone receptor antagonist employed comprises Form L eplerenone.

In another embodiment of the present invention, the aldosterone receptor antagonist employed comprises Form H eplerenone.

While the invention has been described with respect to specific examples including presently preferred modes of carrying out the invention, those skilled in the art will appreciate that there are numerous variations and permutations of the above described systems and techniques that fall within the spirit and scope of the invention.

### ADDITIONAL EXEMPLARY EMBODIMENTS

Additional embodiments are as follows:

1. A method for the prophylaxis or treatment of a cardiovascular-related condition, the method comprising administering to a subject in need thereof, susceptible to or afflicted with such condition, a first amount of an aldosterone receptor antagonist and a second amount of an anti-diabetic agent,
wherein the first amount of the aldosterone receptor antagonist and the second amount of the anti-diabetic agent together comprise a therapeutically-effective amount of the aldosterone receptor antagonist and anti-diabetic agent.

2. The method of Embodiment 1 wherein the cardiovascular-related condition is selected from the group consisting of coronary artery disease, hypertension, cardiovascular disease, renal dysfunction, cerebrovascular disease, vascular disease, retinopathy, neuropathy, hyperglycemia, hyperinsulinemia, insulin resistance, edema, endothelial dysfunction, and baroreceptor dysfunction.

3. The method of Embodiment 1 wherein the cardiovascular-related condition is hypertension.

4. The method of Embodiment 1 wherein the cardiovascular-related condition is cardiovascular disease.

5. The method of Embodiment 4 wherein the cardiovascular disease is selected from the group consisting of coronary artery disease, heart failure, arrhythmia, diastolic dysfunction, systolic dysfunction, ischemia, sudden cardiac death, myocardial fibrosis, vascular fibrosis, impaired arterial compliance, myocardial necrotic lesions, vascular damage, myocardial infarction, left ventricular hypertrophy, decreased ejection fraction, cardiac lesions, vascular wall hypertrophy, endothehal thickening, and fibrinoid necrosis of coronary arteries.

6. The method of Embodiment 4 wherein the cardiovascular disease is heart failure.

7. The method of Embodiment 1 wherein the cardiovascular-related condition is renal dysfunction.

8. The method of Embodiment 7 wherein the renal dysfunction is selected from the group consisting of glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary cells, swelling and proliferation of extracapillary cells, expansion of reticulated mesangial matrix with or without significant hypercellularity, and malignant nephrosclerosis.

9. The method of Embodiment 1 wherein the cardiovascular-related condition is cerebrovascular disease.

10. The method of Embodiment 9 wherein the cerebrovascular disease is stroke.

11. The method of Embodiment 1 wherein the cardiovascular-related condition is vascular disease.

12. The method of Embodiment 11 wherein the vascular disease is selected from the group consisting of thrombotic vascular disease, proliferative arteriopathy, atherosclerosis, decreased vascular compliance, and endothelial dysfunction.

13. The method of Embodiment 1 wherein the cardiovascular-related condition is edema.

14. The method of Embodiment 13 wherein the edema is selected from the group consisting of peripheral tissue edema, hepatic congestion, splenic congestion, liver ascites, respiratory congestion, and lung congestion.

15. The method of Embodiment 1 wherein the cardiovascular-related condition is hyperglycemia, hyperinsulinemia insulin resistance.

16. The method of Embodiment 15 wherein the hyperglycemia, hyperinsulinemia or insulin resistance is selected from the group consisting of insulin resistance, Type I diabetes mellitus, Type II diabetes mellitus, glucose resistance, pre-diabetic state, and metabolic syndrome.

17. The method of Embodiment 1 wherein the cardiovascular-related condition is selected from the group consisting of coronary heart disease, hypertension, cardiovascular disease, stroke, and Type II diabetes mellitus.

18. The method of Embodiment 17 wherein the cardiovascular-related condition is selected from the group consisting of coronary heart disease, hypertension, heart failure, left ventricular hypertrophy and stroke.

19. The method of Embodiment 1 wherein the aldosterone receptor antagonist is an epoxy-steroidal-type compound characterized in having a 9α-, 11α-substituted epoxy moiety.

20. The method of Embodiment 1 wherein the aldosterone receptor antagonist is eplerenone.

21. The method of Embodiment 1 wherein the aldosterone receptor antagonist is a spirolactone-type compound.

22. The method of Embodiment 1 wherein the aldosterone receptor antagonist is spironolactone.

23. The method of Embodiment 1 wherein the aldosterone receptor antagonist is selected from the group consisting of:
pregn-4-ene-7,21-dicarboxylic acid, 9,11-epoxy-17-hydroxy-3-oxo,g-lactone, methyl ester, (7a,11a,17a)-;
pregn-4-ene-7,21-dicarboxylic acid, 9,11-epoxy-17-hydroxy-3-oxo-dimethyl ester,(7a,11a,17a)-;
3′H-cyclopropa(6,7)pregna-4,6-diene-21-carboxylic acid,9,11-epoxy-6,7-dihydro-17-hydroxy-3-oxo-,g-lactone, (6b,7b,11b,17b)-;
pregn-4-ene-7,21-dicarboxylic acid, 9,11-epoxy-17-hydroxy-3-oxo-,7-(1-methylethyl)ester, monopotassium salt,(7a,11a,17a)-;
pregn-4-ene-7,21-dicarboxylic acid, 9,11,-epoxy-17-hydroxy-3-oxo-,7-methyl ester, monopotassium salt, (7a,11a,17a)-;
3′H-cyclopropa(6,7)pregna-1,4,6-triene-21-carboxylic acid, 9,11-epoxy-6,7-dihydro-17-hydroxy-3-oxo-,g-actone(6a,7a,11.a)-;
3′H-cyclopropa(6,7)pregna-4,6-diene-21-carboxylic acid, 9,11-epoxy-6,7-dihydro-17-hydroxy-3-oxo-, methyl ester, (6a,7a,11a,17a)-;
3′H-cyclopropa(6,7)pregna-4,6-diene-21-carboxylic acid, 9,11-epoxy-6,7-dihydro-17-hydroxy-3-oxo-, monopotassium salt, (6a,7a,11.a,17a)-;
3′H-cyclopropa(6,7)pregna-4,6-diene-21-carboxylic acid, 9,11-epoxy-6,7-dihydro-17-hydroxy-3-oxo-, g-lactone, (6a,7a,11a.,17a)-;
pregn-4-ene-7,21-dicarboxylic acid, 9,11-epoxy-17-hydroxy-3-oxo-,g-lactone, ethyl ester, (7a,11a,17a)-; and
pregn-4-ene-7,21-dicarboxylic acid, 9,11-epoxy-17-hydroxy-3-oxo-,g-lactone, 1-methylethyl ester, (7a,11a,17a)-.

24. The method of Embodiment 1 wherein the anti-diabetic agent is selected from the group consisting of Acarbose; Acetohexamide; Buformin; 1-Butyl-3-metanilylurea; Carbutamide; Chlorpropamide; Ciglitazone; Glibornuride; Gliclazide; Glimepiride; Glipizide; Gliquidone; Glisoxepid; Glyburide; Glybuthiazole; Glybuzole; Glyhexamide; Glymidine; Glypinamide; Metformin; Miglitol; Nateglinide; Phenbutamide; Phenformin; Pioglitazone; Proinsulin; Repaglinide; Rosiglitazone; Tolazamide; Tolbutamde; Tolcyclamide; Troglitazone, and pharmaceutically acceptable salts, esters, conjugate acids, and prodrugs thereof.

25. The method of Embodiment 24 wherein the aldosterone receptor antagonist is eplerenone.

26. The method of Embodiment 1 wherein the anti-diabetic agent is Metformin or pharmaceutically acceptable salts, esters, conjugate acids, or prodrugs thereof.

27. The method of Embodiment 26 wherein the aldosterone receptor antagonist is eplerenone.

28. The method of Embodiment 1 wherein the anti-diabetic agent is a sulfonylurea or pharmaceutically acceptable salts, esters, conjugate acids, or prodrugs thereof.

29. The method of Embodiment 28 wherein the aldosterone receptor antagonist is eplerenone.

30. The method of Embodiment 1 wherein the anti-diabetic agent is a PPAR gamma agonist, or pharmaceutically acceptable salts, esters, conjugate acids, or prodrugs thereof.

31. The method of Embodiment 30 wherein the aldosterone receptor antagonist is eplerenone.

32. The method of Embodiment 1 wherein the anti-diabetic agent is an injectable insulin or pharmaceutically acceptable salts, esters, conjugate acids, or prodrugs thereof.

33. The method of Embodiment 32 wherein the aldosterone receptor antagonist is eplerenone.

34. The method of Embodiment 1 wherein the anti-diabetic agent is a Meglitinide analog or other non-sulfonylurea insulin secretagogue.

35. The method of Embodiment 34 wherein the aldosterone receptor antagonist is eplerenone.

36. The method of Embodiment 1 wherein the anti-diabetic agent is selected from the group consisting of agonists of GLP-1 receptors, DPP-IV inhibitors, PPARalpha/gamma dual agonists, inhaled insulins, oral insulins, PTP-1B inhibitors, and fructose-1,6-bisphosphatase inhibitors and the pharmaceutically acceptable salts, esters, conjugate acids, and prodrugs thereof.

37. The method of Embodiment 36 wherein the aldosterone receptor antagonist is eplerenone.

38. The method of Embodiment 1 wherein the anti-diabetic agent is selected from the group consisting of glucocorticoid antagonists, glucagon antagonists, adiponectin/APM1/acrp30 and related analogs, 11-beta-hydroxysteroid dehydrogenase-1 inhibitors, and insulin receptor activators and the pharmaceutically acceptable salts, esters, conjugate acids, and prodrugs thereof.

39. The method of Embodiment 38 wherein the aldosterone receptor antagonist is eplerenone.

40. The method of Embodiment 1 wherein the aldosterone receptor antagonist and the anti-diabetic agent are administered in a sequential manner.

41. The method of Embodiment 1 wherein the aldosterone receptor antagonist and the anti-diabetic agent are administered substantially simultaneously.

42. The method of Embodiment 1 wherein the aldosterone receptor antagonist is administered in a daily dose ranging from about 0.1 to 2000 mg, and the anti-diabetic agent is administered in a daily dose ranging from about 0.1 to 1000 mg.

43. The method of Embodiment 1 wherein the first amount of the aldosterone receptor antagonist produces no substantial diuretic or anti-hypertensive effect in a subject.

44. The method of Embodiment 1 further comprising administering a third amount of a compound selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesterol ester transfer protein inhibitors, bile acid sequestrants, anti-oxidants, vitamin E, probucol, IIbIIa antagonists, xemilofiban, and orbofiban.

45. The method of Embodiment 1 further comprising administering a third amount of an angiotensin converting enzyme inhibitor.

46. The method of Embodiment 45 wherein the aldosterone receptor antagonist is selected from the group consisting of eplerenone and spironolactone.

47. The method of Embodiment 45 wherein the aldosterone receptor antagonist is eplerenone.

48. The method of Embodiment 45 wherein the aldosterone receptor antagonist is spironolactone.

49. The method of Embodiment 45 wherein the anti-diabetic agent is selected from the group consisting of Acarbose; Acetohexamide; Buformin; 1-Butyl-3-metanilylurea; Carbutamide; Chlorpropamide; Ciglitazone; Glibornuride; Gliclazide; Glimepiride; Glipizide; Gliquidone; Glisoxepid; Glyburide; Glybuthiazole; Glybuzole; Glyhexamide; Glymidine; Glypinamide; Metformin; Miglitol; Nateglinide; Phenbutamide; Phenformin; Pioglitazone; Proinsulin; Repaglinide; Rosiglitazone; Tolazamide; Tolbutamde; Tolcyclamide; Troglitazone, and the pharmaceutically acceptable salts, esters, conjugate acids, and prodrugs thereof.

50. The method of Embodiment 45 wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

51. The method of Embodiment 45, wherein the anti-diabetic agent is selected from the group consisting of Acarbose; Acetohexamide; Buformin; 1-Butyl-3-metanilylurea; Carbutamide; Chlorpropamide; Ciglitazone; Glibornuride; Gliclazide; Glimepiride; Glipizide; Gliquidone; Glisoxepid; Glyburide; Glybuthiazole; Glybuzole; Glyhexamide; Glymidine; Glypinamide; Metformin; Miglitol; Nateglinide; Phenbutamide; Phenformin; Pioglitazone; Proinsulin; Repaglinide; Rosiglitazone; Tolazamide; Tolbutamde; Tolcyclamide; Troglitazone, and the pharmaceutically acceptable salts, esters, conjugate acids, and prodrugs thereof, and
wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

52. The method of embodiment 51 wherein the aldosterone receptor antagonist is eplerenone.

53. The method of embodiment 51 wherein the aldosterone receptor antagonist is spironolactone.

54. The method of Embodiment 45 wherein the aldosterone receptor antagonist, anti-diabetic agent, and angiotensin converting enzyme inhibitor are administered in a sequential manner.

55. The method of Embodiment 45 wherein the aldosterone receptor antagonist, anti-diabetic agent, and angiotensin converting enzyme inhibitor are administered in a substantially simultaneous manner.

56. The method of Embodiment 45 wherein the aldosterone receptor antagonist is administered in a daily dose ranging from about 0.1 to 2000 mg, the anti-diabetic agent is administered in a daily dose ranging from about 0.1 to 1000 mg, and the angiotensin converting enzyme inhibitor is administered in a daily dose ranging from about 0.1 to 1000 mg.

57. The method of Embodiment 45 wherein the first amount of the aldosterone receptor antagonist produces no substantial diuretic or anti-hypertensive effect in a subject.

58. A combination comprising an aldosterone receptor antagonist and an anti-diabetic agent.

59. The combination of Embodiment 58 wherein the aldosterone receptor antagonist is eplerenone.

60. The combination of Embodiment 58 wherein the aldosterone receptor antagonist is spironolactone.

61. A pharmaceutical composition comprising a first amount of an aldosterone receptor antagonist, a second amount of an anti-diabetic agent, and a pharmaceutically acceptable carrier.

62. The composition of Embodiment 61 wherein the first amount of the aldosterone receptor antagonist and the second amount of the anti-diabetic agent together comprise a therapeutically-effective amount of the aldosterone receptor antagonist and anti-diabetic agent.

63. The composition of Embodiment 61 wherein the aldosterone receptor antagonist is an epoxy-steroidal-type compound characterized in having a 9α-, 11α-substituted epoxy moiety.

64. The composition of Embodiment 61 wherein the aldosterone receptor antagonist is eplerenone.

65. The composition of Embodiment 61 wherein the aldosterone receptor antagonist is a spirolactone-type compound.

66. The composition of Embodiment 61 wherein the aldosterone receptor antagonist is spironolactone.

67. The composition of Embodiment 61 wherein the aldosterone receptor antagonist is selected from the group consisting of:
pregn-4-ene-7,21-dicarboxylic acid, 9,11-epoxy-17-hydroxy-3-oxo,g-lactone, methyl ester, (7a,11a,17a)-;
pregn-4-ene-7,21-dicarboxylic acid, 9,11-epoxy-17-hydroxy-3-oxo-dimethyl ester,(7a,11a,17a)-;
3′H-cyclopropa(6,7)pregna-4,6-diene-21-carboxylic acid, 9,11-epoxy-6,7-dihydro-17-hydroxy-3-oxo-,g-lactone, (6b,7b,11b,17b)-;
pregn-4-ene-7,21-dicarboxylic acid, 9,11-epoxy-17-hydroxy-3-oxo-,7-(1-methylethyl)ester, monopotassium salt,(7a,11a,17a)-;
pregn-4-ene-7,21-dicarboxylic acid, 9,11,-epoxy-17-hydroxy-3-oxo-,7-methyl ester, monopotassium salt, (7a,11a,17a)-;
3′H-cyclopropa(6,7)pregna-1,4,6-triene-21-carboxylic acid, 9,11-epoxy-6,7-dihydro-17-hydroxy-3-oxo-,g-actone(6a,7a,11.a)-;
3′H-cyclopropa(6,7)pregna-4,6-diene-21-carboxylic acid, 9,11-epoxy-6,7-dihydro-17-hydroxy-3-oxo-, methyl ester, (6a,7a,11a,17a)-;
3′H-cyclopropa(6,7)pregna-4,6-diene-21-carboxylic acid, 9,11-epoxy-6,7-dihydro-17-hydroxy-3-oxo-, monopotassium salt, (6a,7a,11a,17a)-;
3′H-cyclopropa(6,7)pregna-4,6-diene-21-carboxylic acid, 9,11-epoxy-6,7-dihydro-17-hydroxy-3-oxo-, g-lactone, (6a,7a,11a.,17a)-;
pregn-4-ene-7,21-dicarboxylic acid, 9,11-epoxy-17-hydroxy-3-oxo-,g-lactone, ethyl ester, (7a,11a,17a)-; and
pregn-4-ene-7,2 1-dicarboxylic acid, 9,11-epoxy-17-hydroxy-3-oxo-,g-lactone, 1-methylethyl ester, (7a,11a,17a)-.

68. The composition of Embodiment 61 wherein the anti-diabetic agent is selected from the group consisting of Acarbose; Acetohexamide; Buformin; 1-Butyl-3-metanilylurea; Carbutamide; Chlorpropamide; Ciglitazone; Glibornuride; Gliclazide; Glimepiride; Glipizide; Gliquidone; Glisoxepid; Glyburide; Glybuthiazole; Glybuzole; Glyhexamide; Glymidine; Glypinamide; Metformin; Miglitol; Nateglinide; Phenbutamide; Phenformin; Pioglitazone; Proinsulin; Repaglinide; Rosiglitazone; Tolazamide; Tolbutamde; Tolcyclamide; Troglitazone, and the pharmaceutically acceptable salts, esters, conjugate acids, and prodrugs thereof.

69. The composition of Embodiment 68 wherein the aldosterone receptor antagonist is eplerenone.

70. The composition of Embodiment 61 wherein the anti-diabetic agent is Metformin or pharmaceutically acceptable salts, esters, conjugate acids, or prodrugs thereof.

71. The composition of Embodiment 70 wherein the aldosterone receptor antagonist is eplerenone.

72. The composition of Embodiment 61 wherein the anti-diabetic agent is a sulfonylurea or pharmaceutically acceptable salts, esters, conjugate acids, or prodrugs thereof.

73. The composition of Embodiment 72 wherein the aldosterone receptor antagonist is eplerenone.

74. The composition of Embodiment 61 wherein the anti-diabetic agent is a PPAR gamma agonist, or pharmaceutically acceptable salts, esters, conjugate acids, or prodrugs thereof.

75. The composition of Embodiment 74 wherein the aldosterone receptor antagonist is eplerenone.

76. The composition of Embodiment 61 wherein the anti-diabetic agent is an injectable insulin or pharmaceutically acceptable salts, esters, conjugate acids, and prodrugs thereof.

77. The composition of Embodiment 76 wherein the aldosterone receptor antagonist is eplerenone.

78. The composition of Embodiment 61 wherein the anti-diabetic agent is a Meglitinide analog or other non-sulfonylurea insulin secretagogue.

79. The composition of Embodiment 78 wherein the aldosterone receptor antagonist is eplerenone.

80. The composition of Embodiment 61 wherein the anti-diabetic agent is selected from the group consisting of agonists of GLP-1 receptors, DPP-IV inhibitors, PPARalpha/gamma dual agonists, inhaled insulins, oral insulins, PTP-1B inhibitors, and fructose-1,6-bisphosphatase inhibitors and the pharmaceutically acceptable salts, esters, conjugate acids, and prodrugs thereof.

81. The composition of Embodiment 80 wherein the aldosterone receptor antagonist is eplerenone.

82. The composition of Embodiment 61 wherein the anti-diabetic agent is selected from the group consisting of glucocorticoid antagonists, glucagon antagonists, adiponectin/APM1/acrp30 and related analogs, 11-beta-hydroxysteroid dehydrogenase-1 inhibitors, and insulin receptor activators and the pharmaceutically acceptable salts, esters, conjugate acids, and prodrugs thereof.

83. The composition of Embodiment 82 wherein the aldosterone receptor antagonist is eplerenone.

84. The composition of Embodiment 61 wherein the first amount of the aldosterone receptor antagonist produces no substantial diuretic or anti-hypertensive effect in a subject.

85. The composition of Embodiment 61 further comprising a third amount of a compound selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, IIbIIIa antagonists, xemilofiban, and orbofiban.

86. The composition of Embodiment 61 further comprising administering a third amount of an angiotensin converting enzyme inhibitor.

87. The composition of Embodiment 86 wherein the aldosterone receptor antagonist is selected from the group consisting of eplerenone and spironolactone.

88. The composition of Embodiment 86 wherein the aldosterone receptor antagonist is eplerenone.

89. The composition of Embodiment 86 wherein the aldosterone receptor antagonist is spironolactone.

90. The composition of Embodiment 86 wherein the anti-diabetic agent is selected from the group consisting of Acarbose; Acetohexamide; Buformin; 1-Butyl-3-metanilylurea; Carbutamide; Chlorpropamide; Ciglitazone; Glibornuride; Gliclazide; Glimepiride; Glipizide; Gliquidone; Glisoxepid; Glyburide; Glybuthiazole; Glybuzole; Glyhexamide; Glymidine; Glypinamide; Metformin; Miglitol; Nateglinide; Phenbutamide; Phenformin; Pioglitazone; Proinsulin; Repaglinide; Rosiglitazone; Tolazamide; Tolbutamde; Tolcyclamide; Troglitazone, and the pharmaceutically acceptable salts, esters, conjugate acids, and prodrugs thereof.

91. The composition of Embodiment 86 wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

92. The composition of Embodiment 86,
wherein the anti-diabetic agent is selected from the group consisting of Acarbose; Acetohexamide; Buformin; 1-Butyl-3-metanilylurea; Carbutamide; Chlorpropamide; Ciglitazone; Glibornuride; Gliclazide; Glimepiride; Glipizide; Gliquidone; Glisoxepid; Glyburide; Glybuthiazole; Glybuzole; Glyhexamide; Glymidine; Glypinamide; Metformin; Miglitol; Nateglinide; Phenbutamide; Phenformin; Pioglitazone; Proinsulin; Repaglinide; Rosiglitazone; Tolazamide; Tolbutamde; Tolcyclamide; Troglitazone, and the pharmaceutically acceptable salts, esters, conjugate acids, and prodrugs thereof, and
wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

93. The composition of embodiment 92 wherein the aldosterone receptor antagonist is eplerenone.

94. The composition of embodiment 92 wherein the aldosterone receptor antagonist is spironolactone.

95. A kit containing a first amount of an aldosterone receptor antagonist and a second amount of an anti-diabetic agent.

96. The kit of Embodiment 95 comprising the first amount of the aldosterone receptor antagonist in a unit dosage form, and the second amount of an anti-diabetic agent in a unit dosage form.

97. The kit of Embodiment 95 wherein the aldosterone receptor antagonist is an epoxy-steroidal-type compound characterized in having a 9α-, 11α-substituted epoxy moiety.

98. The kit of Embodiment 95 wherein the aldosterone receptor antagonist is eplerenone.

99. The kit of Embodiment 95 wherein the aldosterone receptor antagonist is a spirolactone-type compound.

100. The kit of Embodiment 95 wherein the aldosterone receptor antagonist is spironolactone.

101. The kit of Embodiment 95 wherein the aldosterone receptor antagonist is selected from the group consisting of:
pregn-4-ene-7,21-dicarboxylic acid, 9,11-epoxy-17-hydroxy-3-oxo,g-lactone, methyl ester, (7a,11a,17a)-;
pregn-4-ene-7,21-dicarboxylic acid, 9,11-epoxy-17-hydroxy-3-oxo-dimethyl ester,(7a,11a,17a)-;
3′H-cyclopropa(6,7)pregna-4,6-diene-21-carboxylic acid, 9,11-epoxy-6,7-dihydro-17-hydroxy-3-oxo-,g-lactone, (6b,7b,11b,17b)-;
pregn-4-ene-7,21-dicarboxylic acid, 9,11-epoxy-17-hydroxy-3-oxo-,7-(1-methylethyl)ester, monopotassium salt,(7a,11a,17a)-;
pregn-4-ene-7,21-dicarboxylic acid, 9,11,-epoxy-17-hydroxy-3-oxo-,7-methyl ester, monopotassium salt, (7a,11a,17a)-;
3'H-cyclopropa(6,7)pregna-1,4,6-triene-21-carboxylic acid, 9,11-epoxy-6,7-dihydro-17-hydroxy-3-oxo-,g-actone(6a,7a,11.a)-;
3′H-cyclopropa(6,7)pregna-4,6-diene-21-carboxylic acid, 9,11-epoxy-6,7-dihydro-17-hydroxy-3-oxo-, methyl ester, (6a,7a,11a,17a)-;
3'H-cyclopropa(6,7)pregna-4,6-diene-21-carboxylic acid, 9,11-epoxy-6,7-dihydro-17-hydroxy-3-oxo-, monopotassium salt, (6a,7a,11a,17a)-;
3'H-cyclopropa(6,7)pregna-4,6-diene-21-carboxylic acid, 9,11-epoxy-6,7-dihydro-17-hydroxy-3-oxo-, g-lactone, (6a,7a,11a.,17a)-;
pregn-4-ene-7,21-dicarboxylic acid, 9,11-epoxy-17-hydroxy-3-oxo-,g-lactone, ethyl ester, (7a,11a,17a)-; and
pregn-4-ene-7,21-dicarboxylic acid, 9,11-epoxy-17-hydroxy-3-oxo-,g-lactone, 1-methylethyl ester, (7a,11a,17a)-.

102. The kit of Embodiment 95 wherein the anti-diabetic agent is selected from the group consisting of Acarbose; Acetohexamide; Buformin; 1-Butyl-3-metanilylurea; Carbutamide; Chlorpropamide; Ciglitazone; Glibornuride; Gliclazide; Glimepiride; Glipizide; Gliquidone; Glisoxepid; Glyburide; Glybuthiazole; Glybuzole; Glyhexamide; Glymidine; Glypinamide; Metformin; Miglitol; Nateglinide; Phenbutamide; Phenformin; Pioglitazone; Proinsulin; Repaglinide; Rosiglitazone; Tolazamide; Tolbutamde; Tolcyclamide; Troglitazone, and the pharmaceutically acceptable salts, esters, conjugate acids, and prodrugs thereof.

103. The kit of Embodiment 102 wherein the aldosterone receptor antagonist is eplerenone.

104. The kit of Embodiment 95 wherein the anti-diabetic agent is Metformin or pharmaceutically acceptable salts, esters, conjugate acids, or prodrugs thereof.

105. The kit of Embodiment 104 wherein the aldosterone receptor antagonist is eplerenone.

106. The kit of Embodiment 95 wherein the anti-diabetic agent is a sulfonylurea or pharmaceutically acceptable salts, esters, conjugate acids, or prodrugs thereof.

107. The kit of Embodiment 106 wherein the aldosterone receptor antagonist is eplerenone.

108. The kit of Embodiment 95 wherein the anti-diabetic agent is a PPAR gamma agonist or pharmaceutically acceptable salts, esters, conjugate acids, or prodrugs thereof.

109. The kit of Embodiment 108 wherein the aldosterone receptor antagonist is eplerenone.

110. The kit of Embodiment 95 wherein the anti-diabetic agent is an injectable insulin or pharmaceutically acceptable salts, esters, conjugate acids, or prodrugs thereof.

111. The kit of Embodiment 110 wherein the aldosterone receptor antagonist is eplerenone.

112. The kit of Embodiment 95 wherein the anti-diabetic agent is a Meglitinide analog or other non-sulfonylurea insulin secretagogue.

113. The kit of Embodiment 112 wherein the aldosterone receptor antagonist is eplerenone.

114. The kit of Embodiment 95 wherein the anti-diabetic agent is selected from the group consisting of agonists of GLP-1 receptors, DPP-IV inhibitors, PPARalpha/gamma dual agonists, inhaled insulins, oral insulins, PTP-1B inhibitors, and fructose-1,6-bisphosphatase inhibitors and the pharmaceutically acceptable salts, esters, conjugate acids, and prodrugs thereof.

115. The kit of Embodiment 114 wherein the aldosterone receptor antagonist is eplerenone.

116. The kit of Embodiment 95 wherein the anti-diabetic agent is selected from the group consisting of glucocorticoid antagonists, glucagon antagonists, adiponectin/APM1/acrp30 and related analogs, 11-beta-hydroxysteroid dehydrogenase-1 inhibitors, and insulin receptor activators and the pharmaceutically acceptable salts, esters, conjugate acids, and prodrugs thereof.

117. The kit of Embodiment 116 wherein the aldosterone receptor antagonist is eplerenone.

118. The kit of Embodiment 95 further comprising a third amount of an angiotensin converting enzyme inhibitor.

119. The kit of Embodiment 118 wherein the aldosterone receptor antagonist is selected from the group consisting of eplerenone and spironolactone.

120. The kit of Embodiment 118 wherein the aldosterone receptor antagonist is eplerenone.

121. The kit of Embodiment 118 wherein the aldosterone receptor antagonist is spironolactone.

122. The kit of Embodiment 118 wherein the anti-diabetic agent is selected from the group consisting of Acarbose; Acetohexamide; Buformin; 1-Butyl-3-metanilylurea; Carbutamide; Chlorpropamide; Ciglitazone; Glibornuride; Gliclazide; Glimepiride; Glipizide; Gliquidone; Glisoxepid; Glyburide; Glybuthiazole; Glybuzole; Glyhexamide; Glymidine; Glypinamide; Metformin; Miglitol; Nateglinide; Phenbutamide; Phenformin; Pioglitazone; Proinsulin; Repaglinide; Rosiglitazone; Tolazamide; Tolbutamde; Tolcyclamide; Troglitazone, and the pharmaceutically acceptable salts, esters, conjugate acids, and prodrugs thereof.

123. The kit of Embodiment 118 wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

124. The kit of Embodiment 118, wherein the anti-diabetic agent is selected from the group consisting of Acarbose; Acetohexamide; Buformin; 1-Butyl-3-metanilylurea; Carbutamide; Chlorpropamide; Ciglitazone; Glibornuride; Gliclazide; Glimepiride; Glipizide; Gliquidone; Glisoxepid; Glyburide; Glybuthiazole; Glybuzole; Glyhexamide; Glymidine; Glypinamide; Metformin; Miglitol; Nateglinide; Phenbutamide; Phenformin; Pioglitazone; Proinsulin; Repaglinide; Rosiglitazone; Tolazamide; Tolbutamde; Tolcyclamide; Troglitazone, and the pharmaceutically acceptable salts, esters, conjugate acids, and prodrugs thereof, and
wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

125. The kit of Embodiment 124 wherein the aldosterone receptor antagonist is eplerenone.

126. The kit of Embodiment 124 wherein the aldosterone receptor antagonist is spironolactone.

### FURTHER EMBODIMENTS

127. A method for the treatment of a cardiovascular-related condition, the method comprising administering to a subject susceptible to or afflicted with such condition a first amount of an aldosterone receptor antagonist and a second amount of an anti-diabetic agent, wherein the first amount of the aldosterone receptor antagonist and the second amount of the anti-diabetic agent together comprise a therapeutically-effective amount of the aldosterone receptor antagonist and anti-diabetic agent.

128. The method of Embodiment 127 wherein the aldosterone receptor antagonist is eplerenone.

129. The method of Embodiment 128 wherein the eplerenone is administered in a daily dose range from about 1 mg to about 250 mg.

130. The method of Embodiment 128 wherein the cardiovascular-related condition is selected from the group consisting of coronary artery disease, hypertension, cardiovascular disease, renal dysfunction, diabetic nephropathy, heart failure, cerebrovascular disease, vascular disease, retinopathy, neuropathy, hyperglycemia, hyperinsulinemia, insulin resistance, edema, endothelial dysfunction, and baroreceptor dysfunction.

131. The method of Embodiment 130 wherein the cardiovascular-related condition is hypertension.

132. The method of Embodiment 130 wherein the cardiovascular-related condition is diabetic nephropathy.

133. The method of Embodiment 130 wherein the cardiovascular-related condition is heart failure.

134. The method of Embodiment 128 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

135. The method of Embodiment 128 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

136. The method of Embodiment 128 wherein the anti-diabetic agent is miglitol.

137. The method of Embodiment 128 wherein the anti-diabetic agent is glipizide.

138. The method of Embodiment 128 wherein the anti-diabetic agent is glyburide.

139. The method of Embodiment 128 wherein the anti-diabetic agent is metformin.

140. The method of Embodiment 127 wherein the aldosterone receptor antagonist is spironolactone.

141. The method of Embodiment 140 wherein the cardiovascular-related condition is selected from the group consisting of coronary artery disease, hypertension, cardiovascular disease, renal dysfunction, diabetic nephropathy, heart failure, cerebrovascular disease, vascular disease, retinopathy, neuropathy, hyperglycemia, hyperinsulinemia, insulin resistance, edema, endothelial dysfunction, and baroreceptor dysfunction.

142. The method of Embodiment 140 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

143. The method of Embodiment 140 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

144. The method of Embodiment 140 wherein the anti-diabetic agent is miglitol.

145. The method of Embodiment 140 wherein the anti-diabetic agent is glipizide.

146. The method of Embodiment 140 wherein the anti-diabetic agent is glyburide.

147. The method of Embodiment 140 wherein the anti-diabetic agent is metformin.

148. The method of Embodiment 127 wherein the aldosterone receptor antagonist and the anti-diabetic agent are administered in a sequential manner.

149. The method of Embodiment 127 wherein the aldosterone receptor antagonist and the anti-diabetic agent are administered in a substantially simultaneous manner.

150. A pharmaceutical composition comprising a fist amount of an aldosterone receptor antagonist, a second amount of an anti-diabetic agent, and a pharmaceutically acceptable carrier.

151. The composition of Embodiment 150 wherein the aldosterone receptor antagonist is eplerenone.

152. The composition of Embodiment 151 wherein the eplerenone is administered in a daily dose range from about 1 mg to about 250 mg.

153. The composition of Embodiment 151 wherein the cardiovascular-related condition is selected from the group consisting of coronary artery disease, hypertension, cardiovascular disease, renal dysfunction, diabetic nephropathy, heart failure, cerebrovascular disease, vascular disease, retinopathy, neuropathy, hyperglycemia, hyperinsulinemia, insulin resistance, edema, endothelial dysfunction, and baroreceptor dysfunction.

154. The method of Embodiment 153 wherein the cardiovascular-related condition is hypertension.

155. The method of Embodiment 153 wherein the cardiovascular-related condition is diabetic nephropathy.

156. The method of Embodiment 153 wherein the cardiovascular-related condition is heart failure.

157. The composition of Embodiment 151 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

158. The composition of Embodiment 151 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof

159. The composition of Embodiment 151 wherein the anti-diabetic agent is miglitol.

160. The composition of Embodiment 151 wherein the anti-diabetic agent is glipizide.

161. The composition of Embodiment 151 wherein the anti-diabetic agent is glyburide.

162. The composition of Embodiment 151 wherein the anti-diabetic agent is metformin.

163. The composition of Embodiment 150 wherein the aldosterone receptor antagonist is spironolactone.

164. The composition of Embodiment 163 wherein the cardiovascular-related condition is selected from the group consisting of coronary artery disease, hypertension, cardiovascular disease, renal dysfunction, diabetic nephropathy, heart failure, cerebrovascular disease, vascular disease, retinopathy, neuropathy, hyperglycemia, hyperinsulinemia, insulin resistance, edema, endothelial dysfunction, and baroreceptor dysfunction.

165. The composition of Embodiment 163 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

166. The composition of Embodiment 163 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

167. The composition of Embodiment 163 wherein the anti-diabetic agent is miglitol.

168. The composition of Embodiment 163 wherein the anti-diabetic agent is glipizide.

169. The composition of Embodiment 163 wherein the anti-diabetic agent is glyburide.

170. The composition of Embodiment 163 wherein the anti-diabetic agent is metformin.

171. A kit containing a first amount of an aldosterone receptor antagonist and a second amount of an anti-diabetic agent.

172. The kit of Embodiment 171 wherein the aldosterone receptor antagonist is eplerenone.

173. The kit of Embodiment 172 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

174. The kit of Embodiment 172 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

175. The kit of Embodiment 171 wherein the aldosterone receptor antagonist is spironolactone.

176. The kit of Embodiment 175 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

177. The kit of Embodiment 175 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

### FURTHER ADDITIONAL EXEMPLARY EMBODIMENTS

178. The use of an aldosterone receptor antagonist for the manufacture of a pharmaceutical composition for co-administration with an anti-diabetic agent for the treatment of a subject susceptible to or afflicted with a cardiovascular-related condition.

179. The use of Claim 178 characterized in that the composition further comprises the anti-diabetic agent, wherein the aldosterone receptor antagonist and the anti-diabetic agent together comprise a therapeutically effective amount of the aldosterone receptor antagonist and the anti-diabetic agent.

180. The use of Embodiment 178 or 179 wherein the aldosterone receptor antagonist is eplerenone.

181. The use of Embodiment 178 or 179 2 wherein the aldosterone receptor antagonist is spironolactone.

182. The use of any of Embodiment 178 to 181 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

183. The use of any of Embodiment 178 to 181 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

184. The use of any of Embodiment 178 to 183 wherein the aldosterone receptor antagonist is administered in a daily dose range from about 1 mg to about 250 mg.

185. The use of any of Embodiment 178 to 184 wherein the cardiovascular-related condition is selected from the group consisting of coronary artery disease, hypertension, cardiovascular disease, renal dysfunction, diabetic nephropathy, heart failure, cerebrovascular disease, vascular disease, retinopathy, neuropathy, hyperglycemia, hyperinsulinemia, insulin resistance, edema, endothelial dysfunction, and baroreceptor dysfunction.

186. A pharmaceutical composition comprising a first amount of an aldosterone receptor antagonist, a second amount of an anti-diabetic agent, and a pharmaceutically acceptable carrier.

187. The composition of Embodiment 186 wherein the aldosterone receptor antagonist is eplerenone.

188. The composition of Embodiment 186 wherein the aldosterone receptor antagonist is spironolactone.

189. The composition of any of Embodiments 186 to 188 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

190. The composition of any of Embodiments 186 to 188 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

191. The composition of any of Embodiments 186 to 190 wherein the aldosterone receptor antagonist is administered in a daily dose range from about 1 mg to about 250 mg.

192. A kit containing a first amount of an aldosterone receptor antagonist and a second amount of an anti-diabetic agent.

All citations to books, magazines, journal articles, patents, or any other publications, etc., recited in this application are expressly incorporated herein by reference.

1. A method for the treatment of a cardiovascular-related condition, the method comprising administering to a subject susceptible to or afflicted with such condition a first amount of an aldosterone receptor antagonist and a second amount of an anti-diabetic agent, wherein the first amount of the aldosterone receptor antagonist and the second amount of the anti-diabetic agent together comprise a therapeutically-effective amount of the aldosterone receptor antagonist and anti-diabetic agent.

2. The method of claim 1 wherein the aldosterone receptor antagonist is eplerenone.

3. The method of claim 2 wherein the eplerenone is administered in a daily dose range from about 1 mg to about 250 mg.

4. The method of claim 2 wherein the cardiovascular-related condition is selected from the group consisting of coronary artery disease, hypertension, cardiovascular disease, renal dysfunction, diabetic nephropathy, heart failure, cerebrovascular disease, vascular disease, retinopathy, neuropathy, hyperglycemia, hyperinsulinemia, insulin resistance, edema, endothelial dysfunction, and baroreceptor dysfunction.

5. The method of claim 4 wherein the cardiovascular-related condition is hypertension.

6. The method of claim 4 wherein the cardiovascular-related condition is diabetic nephropathy.

7. The method of claim 4 wherein the cardiovascular-related condition is heart failure.

8. The method of claim 2 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

9. The method of claim 2 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

10. The method of claim 2 wherein the anti-diabetic agent is miglitol.

11. The method of claim 2 wherein the anti-diabetic agent is glipizide.

12. The method of claim 2 wherein the anti-diabetic agent is glyburide.

13. The method of claim 2 wherein the anti-diabetic agent is metformin.

14. The method of claim 1 wherein the aldosterone receptor antagonist is spironolactone.

15. The method of claim 14 wherein the cardiovascular-related condition is selected from the group consisting of coronary artery disease, hypertension, cardiovascular disease, renal dysfunction, diabetic nephropathy, heart failure, cerebrovascular disease, vascular disease, retinopathy, neuropathy, hyperglycemia, hyperinsulinemia, insulin resistance, edema, endothelial dysfunction, and baroreceptor dysfunction.

16. The method of claim 14 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

17. The method of claim 14 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

18. The method of claim 14 wherein the anti-diabetic agent is miglitol.

19. The method of claim 14 wherein the anti-diabetic agent is glipizide.

20. The method of claim 14 wherein the anti-diabetic agent is glyburide.

21. The method of claim 14 wherein the anti-diabetic agent is metformin.

22. The method of claim 1 wherein the aldosterone receptor antagonist and the anti-diabetic agent are administered in a sequential manner.

23. The method of claim 1 wherein the aldosterone receptor antagonist and the anti-diabetic agent are administered in a substantially simultaneous manner.

24. A pharmaceutical composition comprising a first amount of an aldosterone receptor antagonist, a second amount of an anti-diabetic agent, and a pharmaceutically acceptable carrier.

25. The composition of claim 24 wherein the aldosterone receptor antagonist is eplerenone.

26. The composition of claim 25 wherein the eplerenone is administered in a daily dose range from about 1 mg to about 250 mg.

27. The composition of claim 25 wherein the cardiovascular-related condition is selected from the group consisting of coronary artery disease, hypertension, cardiovascular disease, renal dysfunction, diabetic nephropathy, heart failure, cerebrovascular disease, vascular disease, retinopathy, neuropathy, hyperglycemia, hyperinsulinemia, insulin resistance, edema, endothelial dysfunction, and baroreceptor dysfunction.

28. The method of claim 27 wherein the cardiovascular-related condition is hypertension.

29. The method of claim 27 wherein the cardiovascular-related condition is diabetic nephropathy.

30. The method of claim 27 wherein the cardiovascular-related condition is heart failure.

31. The composition of claim 25 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

32. The composition of claim 25 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

33. The composition of claim 25 wherein the anti-diabetic agent is miglitol.

34. The composition of claim 25 wherein the anti-diabetic agent is glipizide.

35. The composition of claim 25 wherein the anti-diabetic agent is glyburide.

36. The composition of claim 25 wherein the anti-diabetic agent is metformin.

37. The composition of claim 24 wherein the aldosterone receptor antagonist is spironolactone.

38. The composition of claim 37 wherein the cardiovascular-related condition is selected from the group consisting of coronary artery disease, hypertension, cardiovascular disease, renal dysfunction, diabetic nephropathy, heart failure, cerebrovascular disease, vascular disease, retinopathy, neuropathy, hyperglycemia, hyperinsulinemia, insulin resistance, edema, endothelial dysfunction, and baroreceptor dysfunction.

39. The composition of claim 37 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

40. The composition of claim 37 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

41. The composition of claim 37 wherein the anti-diabetic agent is miglitol.

42. The composition of claim 37 wherein the anti-diabetic agent is glipizide.

43. The composition of claim 37 wherein the anti-diabetic agent is glyburide.

44. The composition of claim 37 wherein the anti-diabetic agent is metformin.

45. A kit containing a first amount of an aldosterone receptor antagonist and a second amount of an anti-diabetic agent.

46. The kit of claim 45 wherein the aldosterone receptor antagonist is eplerenone.

47. The kit of claim 46 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

48. The kit of claim 46 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.

49. The kit of claim 45 wherein the aldosterone receptor antagonist is spironolactone.

50. The kit of claim 49 wherein the anti-diabetic agent is selected from the group consisting of alpha-glucosidase inhibitors, biguanides, insulins, meglitinides, sulfonylureas, thiazolidinediones, and pharmaceutically acceptable salts, esters, conjugate acids, and prodrugs thereof.

51. The kit of claim 49 wherein the anti-diabetic agent is selected from the group consisting of miglitol, acarbose, metformin, insulin, nateglinide, repaglinide, tolbutamide, chlorpropamide, tolazamide, acetohexamide, glimepiride, glyburide, glipizide, gliclazide, pioglitazone, rosiglitazone, and pharmaceutically acceptable salts, esters, conjugate acids, and prodrugs thereof.
